SEQUENCE LISTING (84 sequences)

SEQ ID NO: 1 - DNA, 32 bp, Artificial Sequence
  Oligonucleotide PCR-amplification of pE194
tatatacagctggattcacaaaaaataggcac

SEQ ID NO: 2 - DNA, 33 bp, Artificial Sequence
  Oligonucleotide PCR-amplification of pE194
tatatacagctggattatgtcttttgcgcagtc

SEQ ID NO: 3 - DNA, 30 bp, Artificial Sequence
  Oligonucleotide PCR-amplification of functional mRFP cassette
tatatggatccgtaatcagggtatcgaggc

SEQ ID NO: 4 - DNA, 31 bp, Artificial Sequence
  Oligonucleotide PCR-amplification of functional mRFP cassette
tatatggatccctcattaggcgggctactaa

SEQ ID NO: 5 - DNA, 33 bp, Artificial Sequence
  Oligonucleotide PCR-amplification of amyB genomic region
tataggtctcaacccataatgccgtcgcactgg

SEQ ID NO: 6 - DNA, 35 bp, Artificial Sequence
  Oligonucleotide PCR-amplification of amyB genomic region
ccttggtctcgtcgctagaagagcagagaggacgg

SEQ ID NO: 7 - DNA, 39 bp, Artificial Sequence
  Oligonucleotide PCR-amplification of amyB genomic region
ttgagaggtctcagagacattttccctatattttcttcc

SEQ ID NO: 8 - DNA, 35 bp, Artificial Sequence
  Oligonucleotide PCR-amplification of amyB genomic region
gtgaggtctcatgagaccatccgttattgacaagg

SEQ ID NO: 9 - DNA, 20 bp, Artificial Sequence
  Oligonucleotide PCR-amplification of amyB genomic region
ttgcccgaatacaacgacag

SEQ ID NO: 10 - DNA, 21 bp, Artificial Sequence
  Oligonucleotide PCR-amplification of amyB genomic region
caacaacaggctgtctgacgg

SEQ ID NO: 11 - DNA, 2143 bp, Bacillus licheniforms
  P300 DSM641 RMS RMS region and flanking region
aacttctataaatgtaacgatacgatttattgtttcattaaagtcttcctttatttcatgttcccatattcttttaatgttccaatccttttcctcgtaatatttattaacttccttatctctttttttatttctttcgagttttttctcccaatattccgtattactttttggtatattcccgtgtttttcacacgcatgccagaaacaagaatcaatgaatatgactattttatatttctgtattactatatctggactaccgtataatttcttaacattttttcggaatcttattccacggtgccatagttctttagtaaccttatcttctaattttgaacgagatttgattgcctgcatgttttttcttctttgttcttttgaaaccgtgtcagtcatagaagagtcctccaaagccacaataattgtattctataaacgaggaagcaagccctcaagcttaccccctcttagttccttttttgcctacttatttatttgttttcattttcaaattcatcataagaacccatttcacaaaaatcaatggagtaatgttgttcgctgtgtttataaacaattactgagtcaatatttagtctttccagcatttcataggttagaagttctttttcctctaagttcataacttgtcttagtagccattctccaagagcactatttggattagacataagtgctttactattgtcttggcataccttggctgataaaagcgatttgtctggcaagcgtaactgaaaaggtttatcacgagctgggaaaaatgttgggaatacattatgaatccattttggaattggtatataaatctcgttagggtttcgtggtcgacctaaagcattccattggtttagaccgcttttttctggtacatgacgctttgagccacggtctgaaaagagtggaagaataacgtgctcaaggttttcaaaaggattgacagttggtgctggaatttttggaatttcaaagccaaatagtttagccaattcatgataaggattttctaagatttcaacattaatttcttcaataggtttatcagtgataaaacgcttataaagggtgctcttagtgacattaaagctgtattcgtgtagaccgtcttcaaaggtgattgtatttctgttgttacttactttcacatttgtaattgaggagatttcaaccaagtccattggctcttcaaaaataagaattttccctggctttcttgttacacagtggtatatcattgaatcaataccatatgttcttttagtaaattcaattctctcgttacggagagaagcaaccgtgtttattagctcttttggagatttcccacgatacaagtctgagtctttattgaattcagctactttttgaagagtatgaccattaccatgaagaaaagtcttaataccaattccgacacgatttaatgaagcgtcagcagaacagtctgacctccccaagttttcagctccaaatgcttcacaaaaagcattttccacattccttgagaccaaataaggcgagtcactttcagagaacaaattggatagcgaaccagttgagcggagcatttgtttgtatgtagtgcagttgatggctggttgattagtatagaacattatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttagg

SEQ ID NO: 12 - DNA, 1146 bp, Bacillus licheniforms
  CDS Restrictase P300 DSM641
ttatttgttttcattttcaaattcatcataagaacccatttcacaaaaatcaatggagtaatgttgttcgctgtgtttataaacaattactgagtcaatatttagtctttccagcatttcataggttagaagttctttttcctctaagttcataacttgtcttagtagccattctccaagagcactatttggattagacataagtgctttactattgtcttggcataccttggctgataaaagcgatttgtctggcaagcgtaactgaaaaggtttatcacgagctgggaaaaatgttgggaatacattatgaatccattttggaattggtatataaatctcgttagggtttcgtggtcgacctaaagcattccattggtttagaccgcttttttctggtacatgacgctttgagccacggtctgaaaagagtggaagaataacgtgctcaaggttttcaaaaggattgacagttggtgctggaatttttggaatttcaaagccaaatagtttagccaattcatgataaggattttctaagatttcaacattaatttcttcaataggtttatcagtgataaaacgcttataaagggtgctcttagtgacattaaagctgtattcgtgtagaccgtcttcaaaggtgattgtatttctgttgttacttactttcacatttgtaattgaggagatttcaaccaagtccattggctcttcaaaaataagaattttccctggctttcttgttacacagtggtatatcattgaatcaataccatatgttcttttagtaaattcaattctctcgttacggagagaagcaaccgtgtttattagctcttttggagatttcccacgatacaagtctgagtctttattgaattcagctactttttgaagagtatgaccattaccatgaagaaaagtcttaataccaattccgacacgatttaatgaagcgtcagcagaacagtctgacctccccaagttttcagctccaaatgcttcacaaaaagcattttccacattccttgagaccaaataaggcgagtcactttcagagaacaaattggatagcgaaccagttgagcggagcatttgtttgtatgtagtgcagttgatggctggttgattagtatagaacat

SEQ ID NO: 13 - DNA, 1022 bp, Artificial Sequence
  homology regions Fusion of 5-prime and 3-prime regions of restrictase gene with flanking BsaI sites
ggtctcgacccaacttctataaatgtaacgatacgatttattgtttcattaaagtcttcctttatttcatgttcccatattcttttaatgttccaatccttttcctcgtaatatttattaacttccttatctctttttttatttctttcgagttttttctcccaatattccgtattactttttggtatattcccgtgtttttcacacgcatgccagaaacaagaatcaatgaatatgactattttatatttctgtattactatatctggactaccgtataatttcttaacattttttcggaatcttattccacggtgccatagttctttagtaaccttatcttctaattttgaacgagatttgattgcctgcatgttttttcttctttgttcttttgaaaccgtgtcagtcatagaagagtcctccaaagccacaataattgtattctataaacgaggaagcaagccctcaagcttaccccctcttagttccttttttgcctacttatttatatttttcctcctcttttatgcttgtcatttcttctttcagacccaaaaggtagtcagctgatacgttcaatgtttcagctattcttttgaaagtgtccaatgatggagttctattttcactttcatatagtgaccaagtgcttctagtgaccccgactttttcagcgatttggctgggtaataacctacgagcttctcttgcattttgaatacgatttccaaggaaaggtatcatttttgcacctccaagatttgttgttttcagagtatcaccagaacccccgaaaatagtccaaagttagctaacagcaaacaaataaaaataaataagttgtttactcttagcaaacttgttactaaaatttgataaagttattcatttaatccagctcttatgctaaaattgcattagcggacaagcttaatgtttgcaaggaggtataattttgacttatcgagtaggtagtatgtttgctgggataggtggaacttgtttaggctcaggagacc

SEQ ID NO: 14 - DNA, 21 bp, Artificial Sequence
  Oligonucleotide PCR-amplification of restrictase genomic region
gacaatccccttttactgacc

SEQ ID NO: 15 - DNA, 24 bp, Artificial Sequence
  Oligonucleotide PCR-amplification of restrictase genomic region
ctatttcatttgcccagacaatcc

SEQ ID NO: 16 - DNA, 1363 bp, Artificial Sequence
  homology regions Fusion of 5-prime and 3-prime regions of pga genes with flanking BsaI sites
ggtctcgacccgaacactgaaattttagaccgggctgggatatcgagacatatataggggcgtttaatggcgaatacagtaacaatgagaatagtaagaaaaattaaaaatgttaaagtttgatgaattatcattgaaaaaaattaatggctttttaaatcctaggattttaacctaaaatctgaagaaataaggtggatcgaacgactcacaaaatatttggatttgtcaatgaatcccgctttatgctaaaagagattttcattttttgatagatggtctgattgtcataggacggatttgttttgaagagggaacattggtgactttttaacctgttcgaaaagagcgaaaatactaaaagaaaagagacatcccggctgacagcccatttaaaggggattgcggccgggggaaaaaagagatcctgaatccatccttcaacctttcatctgaaatagggagaaaagtacaaaaatcataatgtcgaattttgaaagcgcatacttaaaacgctgacaaaaatctgataggaattaagaactttcgatttccaaaaatatcaataaaaagataggcattaatgactcgggcgaggtgatctttgtcacggaaaatttcgtcgtcttctgttacataatgccgattgtgatttcatagtgaaccctgatcccggttataaaagacctgtgaaaagcggccggtttgaaagggaaacacgacaattttcttaaccggtcagtgtataaagttttatagaaaatcaggaggatatatacatggttttggggttcatgtttattgtattcttttgaagggaataaaaactgacaaatttcgactgaagcaaaatttgaaaatgcatcaccttaccaattcgggatgggaaccgcacctcatgttcatgacctctttagaatatttcccttcatctttttaatccgcgcttaggtgaaaaagctgatcatgctgtgctgagcgtttcttctcgctatgacgctgctgtacatgcaaaaaaagtcctttaaatatcccagttgaatgacgatgaaagaggaaagaagaggaggaacagatcaattgataaaaaaagcggcaaacaaaaagttggttttgttttgtggaattgcggtgctttggatgtctttatttttaacgaatcataatgatgtacgcgccgatacgatcggcgagaaaatagcggaaactgccagacagcttgagggtgcgaaatacagctacggcggagagaagccgaaaacggggtttgactcgtcaggctttgtgcaatatgtgtttcaatcgctcgatattacgcttccgagaacggtaaaggaacaatcgactcttggctcaggagacc

SEQ ID NO: 17 - DNA, 20 bp, Artificial Sequence
  Oligonucleotide PCR-amplification of pga genomic region
aaagccttctcctctctatt

SEQ ID NO: 18 - DNA, 20 bp, Artificial Sequence
  Oligonucleotide PCR-amplification of pga genomic region
ttcttgaaaaagacaaggtc

SEQ ID NO: 19 - DNA, 1027 bp, Artificial Sequence
  homology regions Fusion of 5-prime and 3-prime regions of the aprE gene with flanking BsaI sites
ggtctcgacccgaagttcttttttaacatataggtaaaacaatacgaaaaaaggcgccaagtattgaagaattgcagcagccgcggcatttcccttttcgattgaagcaaaaaacgtatattgaacagtaagcattccaaaaatggaaaatactaaaatcgaacaaatatctgtttttttcttccatatctgacacacatgttgaaaaccgtttttcattgaaacatataacaagagaatgactcccgatgccagaagcctgacagagacaagcgagccggcttcaaccgctcccctttcaaatatgtactgtgcagcgcttcccgataatccccacaatgaagcccctgcaagcaccatcaatacgcctttcacatgagctgatttcatatctttcacccgtttctgtatgcgatatattgcatattttaatagatgatcgacaaggccgcaacctccttcggcaaaaaatgatctcataaaataaatgaatagtattttcataaaatgagctcaataacatattctaacaaatagcatatagaaaaagctagtgtttttagcactagctttttcttcattctgatgaaggttgttcaatattttgaatccgttccatgatcgtcggatggccgtatttaaaaatcttgacgagaaacggcgggtttgcctcgctcagcccggcttttgagagctcttgaaacgtcgaaaccgctgcatcgctgttttgcgtcagttcaatcgcatactggtcagcagctttttcctgatgcctcgaaactgcgttcgtaaatggagacgacgcgaaagagatgacccccatcagcatcagaagaagcggaagtgcggctagatcggattttcctgcaatatgaaggcttcttccatagcggccgatgatccgcttgtacagcttgtcgatcacataaaagacagcaagggataaaagcagatacccgccaagtcctatgtaaacatgcttcatcacatagtgccccatttcgtgcgccatgatgctcaggagacc

SEQ ID NO: 20 - DNA, 21 bp, Artificial Sequence
  Oligonucleotide PCR-amplification of aprE genomic region
ccggttgtcattgatccttta

SEQ ID NO: 21 - DNA, 21 bp, Artificial Sequence
  Oligonucleotide PCR-amplification of aprE genomic region
atcctcctgcaaaaaccgtat

SEQ ID NO: 22 - DNA, 1500 bp, Artificial Sequence
  functional fragment PxylA promoter and xylR repressor Bacillus megaterium flanked by BpiI sites
gaagaccttcgagttatagaaaatcaattaattgaaagtagctccttcattcttaagatcaacgtgatataggtttgctaacctttgcgttcacttaactaacttataggggtaacacttaaaaaagaatcaataacgatagaaaccgctcctaaagcaggtgcattttttcctaacgaagaaggcaatagttcacatttattgtctaaatgagaatggactctagaagaaacttcgtttttaatcgtatttaaaacaatgggatgagattcaattatatgatttctcaagataacagcttctatatcaaatgtattaaggatattggttaatccaattccgatataaaagccaaagttttgaagtgcatttaacatttctacatcatttttatttgcgcgttccacaatctcttttcgagaaatattcttttcttctttagagagcgaagccagtaacgctttttcagaagcatataattcccaacagcctcgatttccacagctgcatttgggtccattaaaatctatcgtcatatgacccatttccccagaaaaaccctgaacacctttatacaattcgttgttaataacaagtccagttccaattccgatattaatactgatgtaaacgatgttttcatagttttttgtcataccaaatactttttcaccgtatgctcctgcattagcttcattttcaacaaaaaccggaacattaaactcactctcaattaaaaactgcaaatctttgatattccaatttaagttaggcatgaaaataatttgctgatgacgatctacaaggcctggaacacaaattcctattccgactagaccataaggggactcaggcatatgggttacaaaaccatgaataagtgcaaataaaatctcttttacttcactagcggaagaactagacaagtcagaagttttctcgagaataatatttccttctaagtcggttagaattccgttaagatagtcgactcctatatcaataccaatcgagtagcctgcattcttattaaaaacaagcattacaggcctcctgccgcctctagattgccctgccccaatttcaaaaataaaatctttttcaagcagtgtatttacttgagaggagacagtagacttgtttaatcctgtaatctcagagagagttgccctggagacaggggagttcttcaaaatttcatctaatattaatttttgattcattttttttactaaagcttgatctgcaatttgaataataaccactcctttgtttatccaccgaactaagttggtgttttttgaagcttgaattagatatttaaaagtatcatatctaatattataactaaattttctaaaaaaaacattgaaataaacatttattttgtatatgatgagataaagttagtttattggataaacaaactaactcaattaagatagttgatggataaacttgttcacttaaatcaaagggggaaatgtaatataagtcttc

SEQ ID NO: 23 - DNA, 813 bp, Artificial Sequence
  functional fragment Cargo vector for type-II- assembly
ttcacctgcgatggcgagaacttgcgaggtctcagcgatcgtctcagcgacccgggtcagctgggtgctttttttattccttcatcagaatgaagaaaaagctagtgctaaaaacactagctttttctatatgctatttgtggcgcgccggctagggtcttcttaattaactcgaggtcgacaagcttactatgctacatcagagcagattgtactgagagtgcaccagatgcggtgtgaaataccgcacagatgcgtaaggagaaaataccgcatcagacgccattcgccattcaggctacgcaactgttgggaagggcgatctgtgcgggcctcttcgctattacgccaactggcgaaagggggatgtgctgcaaggcgattaagttgggtaacgccagggttttcccagtcacgacgttgtaaaacgacggccagtgagcgcgcgtaatacgactcactggaactccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtaagctaactcacattaattgcgttgcgctgatatcgaattccaccgcggtgagctctgtggcatcaaataaaacgaaaagctcagtcgaaagactgggcctttcgttttatctgttgttggatccaactagtgaagaccctcgacaatttgagatgagaccttgcaggtgagatgagacggtccttaacatcgc

SEQ ID NO: 24 - DNA, 21 bp, Artificial Sequence
  Oligonucleotide qPCR terminator Bsub fwd
caagaagctcatgcaaagacc

SEQ ID NO: 25 - DNA, 21 bp, Artificial Sequence
  Oligonucleotide qPCR terminator Bsub rev
cgatgacagagcaagagagac

SEQ ID NO: 26 - DNA, 20 bp, Artificial Sequence
  Oligonucleotide qPCR plasmid AmpR fwd
atcagcaataaaccagccag

SEQ ID NO: 27 - DNA, 19 bp, Artificial Sequence
  Oligonucleotide qPCR plasmid AmpR rev
agccataccaaacgacgag

SEQ ID NO: 28 - DNA, 28 bp, Artificial Sequence
  Oligonucleotide delta cop+repF fwd
atcgccatgggacaatggacacctaggg

SEQ ID NO: 29 - DNA, 30 bp, Artificial Sequence
  Oligonucleotide delta repF rev
atcgccatggctcggcgtatgttattcaag

SEQ ID NO: 30 - DNA, 43 bp, Artificial Sequence
  Oligonucleotide PsrfAA(trunc) fwd
gcattctagagaagacaaatatttttatctttctaccgttcag

SEQ ID NO: 31 - DNA, 41 bp, Artificial Sequence
  Oligonucleotide PsrfAA(trunc) rev
gcatgaattcgaagacgctcgattagtggaaatgattgcgg

SEQ ID NO: 32 - DNA, 40 bp, Artificial Sequence
  Oligonucleotide Hom(luxA) fwd
cagtgaattcggtctctacccgaggtaatgaaacgtttgg

SEQ ID NO: 33 - DNA, 39 bp, Artificial Sequence
  Oligonucleotide Hom(luxA) rev
agcttctagaggtctcttgagcccatattcttgagccac

SEQ ID NO: 34 - DNA, 28 bp, Artificial Sequence
  Oligonucleotide KanR - ColE1 fwd NcoI
tcagccatgggttcaacaaacgggccat

SEQ ID NO: 35 - DNA, 36 bp, Artificial Sequence
  Oligonucleotide KanR - ColE1 rev PstI
tgacctgcagagtacttgagttttcgttccactgag

SEQ ID NO: 36 - DNA, 44 bp, Artificial Sequence
  Oligonucleotide T2Acassette fwd BamHI
tacgagatctgtcgacggatccgatatcccaccctgagacccac

SEQ ID NO: 37 - DNA, 39 bp, Artificial Sequence
  Oligonucleotide T2A cassette rev NcoI
gtacccatgggatatcttaaattgagtgagacgagcagg

SEQ ID NO: 38 - DNA, 28 bp, Artificial Sequence
  Oligonucleotide Terminator fwd SpeI
tagcactagtctaaaaaggagcgatgag

SEQ ID NO: 39 - DNA, 29 bp, Artificial Sequence
  Oligonucleotide Terminator rev BamHI
gatcggatccgtaatctagagcaacgttc

SEQ ID NO: 40 - DNA, 31 bp, Artificial Sequence
  Oligonucleotide pE194 ori fwd SpeI
gatcactagtgacgatttagagatgtagagg

SEQ ID NO: 41 - DNA, 28 bp, Artificial Sequence
  Oligonucleotide pE194 ori rev NcoI
gcatccatgggctggtgcgaaaaaagag

SEQ ID NO: 42 - DNA, 28 bp, Artificial Sequence
  Oligonucleotide oriT fwd NcoI
atgcccatgggactaaatttctgtatgg

SEQ ID NO: 43 - DNA, 26 bp, Artificial Sequence
  Oligonucleotide oriT rev XbaI
gacttctagaccccttgttaacgctc

SEQ ID NO: 44 - DNA, 33 bp, Artificial Sequence
  Oligonucleotide Pxyl_cop fwd NsiI
gatcatgcatcatatgtcgagttatagaaaatc

SEQ ID NO: 45 - DNA, 31 bp, Artificial Sequence
  Oligonucleotide Pxyl_cop rev SpeI
tgacactagtctttttttattccttcatcag

SEQ ID NO: 46 - DNA, 46 bp, Artificial Sequence
  Oligonucleotide Quickchange lux001
gaagatagtaataaagctaaacaagagacacgtgcatttattagtg

SEQ ID NO: 47 - DNA, 36 bp, Artificial Sequence
  Oligonucleotide Quickchange lux002
gatctcatgacggaatgaggccgttgcaacgattag

SEQ ID NO: 48 - DNA, 44 bp, Artificial Sequence
  Oligonucleotide Quickchange lux003
gatgattattgagtcaaatgcagatgtggaatttaatcaaccac

SEQ ID NO: 49 - DNA, 21 bp, Artificial Sequence
  Artificial oligonucleotide
gttatgcatatgcaaaaaaac

SEQ ID NO: 50 - DNA, 18 bp, Artificial Sequence
  Artificial oligonucleotide
cgcgtacgatgacctcta

SEQ ID NO: 51 - DNA, 18 bp, Artificial Sequence
  Artificial oligonucleotide
accttcttcaactaacgg

SEQ ID NO: 52 - DNA, 20 bp, Artificial Sequence
  Artificial oligonucleotide
gaagaaagcagacaagtaag

SEQ ID NO: 53 - DNA, 39 bp, Artificial Sequence
  Oligonucleotide sacA 5-prime hom region fwd
cagtgaattcggtctctacccgccgggcttatgaagaag

SEQ ID NO: 54 - DNA, 41 bp, Artificial Sequence
  Oligonucleotide sacA 5-prime hom region rev
taggcttctagaggtctcttcgcatcgggccaagaaatctc

SEQ ID NO: 55 - DNA, 39 bp, Artificial Sequence
  Oligonucleotide sacA 3-prime hom region fwd
agctgaattcggtctctgagactggattcactgcatgac

SEQ ID NO: 56 - DNA, 39 bp, Artificial Sequence
  Oligonucleotide sacA 3-prime hom region rev
agcttctagaggtctcttgagcactggctgttacttcat

SEQ ID NO: 57 - DNA, 42 bp, Artificial Sequence
  Oligonucleotide rev lux
gcattctagagaagacgcgctagcactggtaatggtagcgac

SEQ ID NO: 58 - DNA, 46 bp, Artificial Sequence
  Oligonucleotide fwd lux
gcatgaattcgaagacgcatatgaaatttggaaactttttgcttac

SEQ ID NO: 59 - DNA, 39 bp, Artificial Sequence
  Oligonucleotide Cop fwd
gcatgaattcgaagacgcatatggttgtagatagaaaag

SEQ ID NO: 60 - DNA, 40 bp, Artificial Sequence
  Oligonucleotide Cop rev
gcattctagagaagacgcgctattaaaatgcaccgtattc

SEQ ID NO: 61 - DNA, 41 bp, Artificial Sequence
  Oligonucleotide fwd lux
gcatgaattcgaagacgcatatgcttttataagtcgacagg

SEQ ID NO: 62 - DNA, 38 bp, Artificial Sequence
  Oligonucleotide Pveg T2A fwd
cgaggaagaccttcgagagttctgagaattggtatgcc

SEQ ID NO: 63 - DNA, 38 bp, Artificial Sequence
  Oligonucleotide Pveg T2A rev
attcgaagacttatatagagtcctcctgtcgaccttac

SEQ ID NO: 64 - DNA, 44 bp, Artificial Sequence
  Oligonucleotide Primer G to A 5-prime of repF
ctatgactaaattttgttaaatgtattagcaccgttattatatc

SEQ ID NO: 65 - DNA, 36 bp, Artificial Sequence
  Oligonucleotide Primer G to A within repF
gggatactgatacagaagataggatgaaaaaagagc

SEQ ID NO: 66 - DNA, 37 bp, Artificial Sequence
  Oligonucleotide amylase gene amyE fwd
gagaggtctcgacccaaatgtgtgcagccgctgaaga

SEQ ID NO: 67 - DNA, 36 bp, Artificial Sequence
  Oligonucleotide amylase gene amyE rev
gagagagaccgtcgccgactctacccatgtcactag

SEQ ID NO: 68 - DNA, 3123 bp, Artificial Sequence
  replication origin functional fragment replication origin pBS72 modified
ggttgcggtctcaccacgcgctagtttgtctgcaactgaaaagtttataccttacctggaacaaatggttgaaacatacgaggctaatatcggcttattaggaatagtccctgtactaataaaatcaggtggatcagttgatcagtatattttggacgaagctcggaaagaatttggagatgacttgcttaattccacaattaaattaagggaaagaataaagcgatttgatgttcaaggaatcacggaagaagatactcatgataaagaagctctaaaactattcaataaccttacaatggaattgatcgaaagggtggaaggttaatggtacgaaaattaggggatctacctagaaagccacaaggcgataggtcaagcttaaagaacccttacatggatcttacagattctgaaagtaaagaaacaacagaggttaaacaaacagaaccaaaaagaaaaaaagcattgttgaaaacaatgaaagttgatgtttcaatccataataagattaaatcgctgcacgaaattctggcagcatccgaagggaattcatattacttagaggatactattgagagagctattgataagatggttgagacattacctgagagccaaaaaactttttatgaatatgaattaaaaaaaagaaccaacaaaggctgagacagactccaaacgagtctgtttttttaaaaaaaatattaggagcattgaatatatattagagaattaagaaagacatgggaataaaaatattttaaatccagtaaaaatatgataagattatttcagaatatgaagaactctgtttgtttttgatgaaaaaacaaacaaaaaaaatccacctaacggaatctcaatttaactaacagcggccaaactgagaagttaaatttgagaaggggaaaaggcggatttatacttgtatttaactatctccattttaacattttattaaaccccatacaagtgaaaatcctcttttacactgttcctttaggtgatcgcggagggacattatgagtgaagtaaacctaaaaggaaatacagatgaattagtgtattatcgacagcaaaccactggaaataaaatcgccaggaagagaatcaaaaaagggaaagaagaagtttattatgttgctgaaacggaagagaagatatggacagaagagcaaataaaaaacttttctttagacaaatttggtacgcatataccttacatagaaggtcattatacaatcttaaataattacttctttgatttttggggctattttttaggtgctgaaggaattgcgctctatgctcacctaactcgttatgcatacggcagcaaagacttttgctttcctagtctacaaacaatcgctaaaaaaatggacaagactcctgttacagttagaggctacttgaaactgcttgaaaggtacggttttatttggaaggtaaacgtccgtaataaaaccaaggataacacagaggaatccccgatttttaagattagacgtaaggttcctttgctttcagaagaacttttaaatggaaaccctaatattgaaattccagatgacgaggaagcgcatgtaaagaaggctttaaaaaaggaaaaagagggacttccaaaggttttgaaaaaagagcacgatgaatttgttaaaaaaatgatggatgagtcagaaacaattaatattccagaggctttacaatatgacacaatgtatgaagatatactcagtaaaggagaaattcgaaaagaaatcaaaaaacaaatacctaatcctacaacatcttttgagagtatatcaatgacaactgaagaggaaaaagtggacagtactttaaaaagcgaaatgcaaaatcgtgtctctaagccttcttttgatacctggtttaaaaacactaagatcaaaattgaaaataaaaattgtttattacttgtaccgagtgaatttgcatttgaatggattaagaaaagatatttagaaacaattaaaacagtccttgaagaagctggatatgttttcgaaaaaatcgaactaagaaaagtgcaataaactgctgaagtatttcagcagttttttttatttagaaatagtgaaaaaaatataatcagggaggtatcaatatttaatgagtactgatttaaatttatttagactggaattaataattaacacgtagactaattaaaatttaatgagggataaagaggatacaaaaatattaatttcaatccctattaaattttaacaagggggggattaaaatttaattagaggtttatccacaagaaaagaccctaataaaatttttactagggttataacactgattaatttcttaatgggggagggattaaaatttaatgacaaagaaaacaatcttttaagaaaagcttttaaaagataataataaaaagagctttgcgattaagcaaaactctttactttttcattgacattatcaaattcatcgatttcaaattgttgttgtatcataaagttaattctgttttgcacaaccttttcaggaatataaaacacatctgaggcttgttttataaactcagggtcgctaaagtcaatgtaacgtagcatatgatatggtatagcttccacccaagttagcctttctgcttcttctgaatgtttttcatatacttccatgggtatctctaaatgattttcctcatgtagcaaggtatgagcaaaaagtttatggaattgatagttcctctctttttcttcaacttttttatctaaaacaaacactttaacatctgagtcaatgtaagcataagatgtttttccagtcataatttcaatcccaaatcttttagacagaaattctggacgtaaatcttttggtgaaagaatttttttatgtagcaatatatccgatacagcaccttctaaaagcgttggtgaatagggcattttacctatctcctctcattttgtggaataaaaatagtcatattcgtccatctacctatcctattatcgaacagttgaactttttaatcaaggatcagtcctttttttcattattcttaaactgtgctcttaactttaacaactcgatttgtttttccagatcccgctcagagacccgttg

SEQ ID NO: 69 - DNA, 1221 bp, Artificial Sequence
  functional fragment Type-II mRFP cassette
ggttgcggtctcaacctttatggttttggtcggcactgccgacagcctcgcagagcacacactttatgaatataaagtatagtgtgttatactttacttggaagtggttgccggaaagagcgaaaatgcctcacatttgtgccacctaaaaaggagcgatttacatatgtcgagggtcttcggtaccttgtcggataaagctgtgttatattatgtcttggtgttaaatacacacgcttaacgatttatgcagagggtgctgcaggcggcagttctgtacaaaaatgacctaagcggaggaaaaaaaccattatattaggaggaaataacatggcctcttcagaggatgttattaaagaatttatgcggtttaaggtgaggatggaaggctcggtgaacggacatgagttcgaaattgagggagaaggtgaaggccgcccttatgaaggtactcagacagcgaaattgaaagtcacgaaaggcggaccgctgccgtttgcttgggacattctctcacctcaatttcaatatggctcaaaagcctacgtaaaacacccggctgacatccctgattacttaaagctatccttcccggagggctttaaatgggaacgagttatgaattttgaggacggcggcgtcgttactgtcacacaggattcttcccttcaggatggcgaatttatttacaaagtaaaacttcgtggaactaacttcccaagcgatggtcccgtgatgcaaaaaaaaacaatgggatgggaagcatctacggaacgtatgtatccggaggatggagccttaaagggtgaaatcaaaatgcgcctgaaacttaaagatggcggacactatgacgcggaagttaaaacaacatatatggctaaaaaaccagtccaactgccgggagcatataagacggatataaagttggacattaccagccataatgaagattacacgattgtggaacagtatgagagagcagagggcagacatagcacaggcgcgtaagaattaatgaaaaataagcggcagcctgcttttccatgcgggctgccgcttatcgggttattgcggccgcgtttataatgaagaccctagccatattctaacaaatagcatatagaaaaagctagtgtttttagcactagctttttcttcattctgatgaaggaataaaaaaagcacctgaaaaggtgtctttttttccacagagacccgttg

SEQ ID NO: 70 - DNA, 1817 bp, Artificial Sequence
  functional fragment modified kanamycin resistance gene and ColE1 replication origin
ggttgcggtctcagctctagttcaacaaacgggccatattgttgtataagtgatgaaatactgaatttaaaacttagtttatatgtggtaaaatgttttaatcaagtttaggaggaattaattatgaagtgtaataatggaccaataataatgactagagaagaaagaatgaagattgttcatgaaattaaggaacgaatattggataaatatggggatgatgttaaggctattggtgtttatggctctcttggtcgtcagactgatgggccctattcggatattgagatgatgtgtgtcatgtcaacagaggaagcagagttcagccatgaatggacaaccggtgagtggaaggtggaagtgaattttgatagcgaagagattctactagattatgcatctcaggtggaatcagattggccgcttacacatggtcaatttttctctattttgccgatttatgattcaggtggatacttagagaaagtgtatcaaactgctaaatcggtagaagcccaaacgttccacgatgcgatttgtgcccttatcgtagaagagctgtttgaatatgcaggcaaatggcgtaatattcgtgtgcaaggaccgacaacatttctaccatccttgactgtacaggtagcaatggcaggtgccatgttgattggtctgcatcatcgcatctgttatacgacgagcgcttcggtcttaactgaagcagttaagcaatcagatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaatcgttaagggatcaactttgggagagagttcaaaattgatcctttttttataacaggaattctgagcaaaaggccagcaaaaggccaggaaccgtaaaaagcgcgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctcgccgttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgacccgtgcgccttatccggtaactatcgtcttgagtccaacccggtaagacacgacttatcgccactggcacgagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaacgagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtacaattcaaatctttttgttccattaaagggcgcgattgctgaatacctagagacccgttg

SEQ ID NO: 71 - DNA, 51 bp, Artificial Sequence
  Oligonucleotide PCR-amplification terminator pMutin
cacatttgtgccacctaaaaaggagcgatgagtcgcttttgtaaatttgga

SEQ ID NO: 72 - DNA, 51 bp, Artificial Sequence
  Oligonucleotide PCR-amplification terminator pMutin
caaggtaccgaagaccctcgacatatgtaatctagagcaacgttcttgcca

SEQ ID NO: 73 - DNA, 22 bp, Artificial Sequence
  Oligonucleotide PCR-amplification pBW424
ttacatatgtcgagggtcttcg

SEQ ID NO: 74 - DNA, 20 bp, Artificial Sequence
  Oligonucleotide PCR-amplification pBW424
atcgctcctttttaggtggc

SEQ ID NO: 75 - DNA, 38 bp, Artificial Sequence
  Oligonucleotide fwd amyB
tcgcgaagacaatcgcgatccgcgacattaacgtttac

SEQ ID NO: 76 - DNA, 37 bp, Artificial Sequence
  Oligonucleotide rev amyB
acccgaagacttacccgattgataatgaagcggcacg

SEQ ID NO: 77 - DNA, 33 bp, Artificial Sequence
  Oligonucleotide fwd primer, T2A cassette from plasmid p#0692 with ter
gcatggtctcatcgaccggaaagagcgaaaatg

SEQ ID NO: 78 - DNA, 34 bp, Artificial Sequence
  Oligonucleotide rev primer; T2A cassette from plasmid p#0692 with ter
cgatggtctctgctacgcgtggaaaaaaagacac

SEQ ID NO: 79 - DNA, 24 bp, Artificial Sequence
  Artificial oligonucleotide
gagattccggtcgacaatttgaga

SEQ ID NO: 80 - DNA, 24 bp, Artificial Sequence
  Artificial oligonucleotide
tgagtctcaaattgtcgaccggaa

SEQ ID NO: 81 - DNA, 21 bp, Artificial Sequence
  Oligonucleotide qPCR luxB fwd
atgtaacagcaaccagtcatc

SEQ ID NO: 82 - DNA, 20 bp, Artificial Sequence
  Oligonucleotide qPCR luxB rev
atccgcaacggctttatatc

SEQ ID NO: 83 - DNA, 22 bp, Artificial Sequence
  Oligonucleotide qPCR ter Bli fwd
gtctaagacatggttccattcg

SEQ ID NO: 84 - DNA, 21 bp, Artificial Sequence
  Oligonucleotide qPCR ter Bli rev
caaactttgcacattgcatcc

(19) United States
(12) Patent Application Publication
(10) Pub. No.: US 2023/0227834 A1
(43) Pub. Date: Jul. 20, 2023

(54) Plasmid Copy Number Regulation and Integration

(71) Applicant: BASF SE, Ludwigshafen am Rhein (DE)

(72) Inventors: Max Fabian Felle, Ludwigshafen (DE); Christopher SAUER, Ludwigshafen (DE); Karen STETTER, Dresden (DE); Diana WOLF, Dresden (DE); Thorsten MASCHER, Dresden (DE)

(21) Appl. No.: 18/002,055

(22) PCT Filed: Jun. 17, 2021
(86) PCT No.: PCT/EP2021/066404

§ 371 Date: Dec. 16, 2022

(30) Foreign Application Priority Data
Jun. 17, 2020 (EP) 20180619.7

(51) Int. Cl. C12N 15/69; C12N 15/90; C12N 15/65

(52) U.S. Cl. CPC C12N 15/69; C12N 15/902; C12N 15/65

(57) ABSTRACT

Described herein are materials and methods for user-controlled adjustment of plasmid copy numbers.

The invention pertains to the field of plasmid copy number adjustment, preferably in prokaryotic hosts, and provides materials and methods for user-controlled adjustment of plasmid copy numbers. The invention furthermore provides host cultivation methods which make use of the means for copy number adjustment provided by the invention. The invention in particular allows to integrate a target nucleic acid segment into another nucleic acid with high integration efficiency.

### BACKGROUND OF THE INVENTION

Plasmids are extrachromosomal genomes that replicate autonomously and in a controlled manner. They generally are circular nucleic acids which carry their own origin of replication to allow them to be replicated independent of the replication of the chromosome of a host microorganism. Plasmids are thus frequently used as vectors to shuttle following nucleic acid segments into a host. Plasmids are generally characterised by their specific plasmid copy number. The replication machinery relied on by a plasmid ensures that the copy number of the plasmid in each host cell does not rise above or fall below the plasmid's characteristic copy number. For example, the well-known plasmid pBR322 has a copy number of approximately 20. This copy number is more or less maintained during all life stages of a host cell.

When a plasmid contains a target gene to be expressed in the host, the plasmid copy number is intricately linked to the gene dosage of the target gene. During fermentation it is generally desirable to achieve a strong expression of the one or more target genes. One way to achieve such strong expression is to put the target gene or genes under the control of a strong promoter. However, for some host microorganisms the available promoters are found to be not strong enough. Another way to increase expression of a target gene is to provide further copies of the promoter-gene expression cassette, thereby increasing gene dosage. The target gene is thus transcribed simultaneously from several locations which increases expression of the target gene. However, increasing the copy number of an expression cassette on a plasmid is laborious and leads to homologous recombination, which in turn can lead to an uncontrolled loss of target gene expression cassettes.

It has therefore been tried to manipulate the copy number of the plasmids themselves. Homologous recombination between identical plasmids, particularly when comprising only one copy of a target gene expression cassette, is less likely to result in loss of the target gene. Several strategies have been proposed to increase plasmid copy numbers. For example,

WO2009076196 and WO2002029067 provide particular high copy number plasmids. WO2007035323 provides plasmids wherein the origin of replication can be exchanged; this allows to insert, into a selected plasmid vector, an origin of replication which is known to provide a desired plasmid copy number.

A high plasmid copy number comes at a price. Generally the size of a target gene expression cassette is small compared to the overall plasmid size. Furthermore, plasmids generally need the presence of a selection marker, typically an antibiotic resistance gene, to ensure maintenance of the plasmid in the host during cultivation. Thus an increase in plasmid copy number results in a high metabolic burden for the host which has to divert resources for the replication of the plasmid backbone. This metabolic burden may become a decisive factor limiting the host's viability under challenging conditions. For example, where the target gene expression already creates a metabolic burden, then the maintenance of a high plasmid copy number may damage the host's viability during fermentation, leading to premature fermentation stop or insufficient yield of the target gene product. Furthermore, when hosts are placed into a new medium, for example when inoculating a fermenter to start a batch fermentation, but also after thawing of cryoconserved cultures, hosts experience strong metabolic stress to adapt to the new environment. In such situations the need to maintain a high plasmid copy number as dictated by the autonomous replication machinery of the plasmid may be too much for the host cell, leading to significant growth delays and thus reduced space-time yields for fermentations.

It has therefore been attempted to restrict plasmid replication of high copy number plasmids for a determined length of time by use of temperature sensitive origin of replications. One example of such temporary restriction of plasmid replication is given in Olson et al, Journal of Biological Engineering 2012, 6:5 and WO9318164. The latter document provides temperature-sensitive plasmid variants of plasmid pWV01 for gram positive bacteria, in particular lactic bacteria, wherein the plasmids are incapable of replication above approximately 37° C. However, in such systems plasmid replication is either completely allowed or completely blocked due to the prevailing temperature. Thus, great care must be taken to prevent accidental plasmid loss at such growth stages where maintenance of a low plasmid copy number (instead of zero plasmids per host cell) is desired.

A further use for plasmids is the manipulation of the non-plasmid genetic material of a host, also called the host chromosome. By way of homologous recombination plasmids are able to integrate into the host's chromosome. They can furthermore be excised from their integration site, again for example by homologous recombination as described in FIGS. 6, 7A and 7B of WO9318164 and the accompanying description thereto. By way of such integration and optional partial excision plasmids can be used for example to inactivate a desired nucleic acid sequence in the hosts chromosome. The inactivation results from the presence of the whole inserted plasmid or the remains thereof after partial plasmid excision. Furthermore, integration of the whole plasmid and/or leaving a segment of plasmid nucleic acid after partial plasmid excision allows to introduce a desired nucleic acid stretch, preferably comprising a target gene expression cassette or introducing any other desired nucleic acid element at a specified location of the hosts chromosome.

Plasmid integration as the first step in such procedure relies on the propensity of the plasmid to recombine with the hosts chromosome instead of continued autonomous replication. Thus it is required first to establish the presence of the plasmid in the host cell. This, in turn, relies on the ability of the plasmid to autonomously replicate. However, when trying to enforce plasmid integration into the host's chromosome, the ability of a plasmid to autonomously replicate severely reduces the likelihood of integration. It has thus been tried to use plasmids with temperature-dependent loss of replicative activity in such integration assays. However, while it may be possible to interfere with the plasmid's ability to reproduce by severely reducing or increasing the temperature of cultivation compared to the host's optimal temperature, such temperature change puts the host cell under severe metabolic stress. For example, at low temperatures host cells may be metabolically more or less in active and at high temperatures host cell proteins may denature. In both cases the likelihood of plasmid integration is reduced. Thus, plasmid integration assays have so far been found to be unreliable and require a lot of work to check that putative clones obtained by such assays are indeed free of plasmids while having integrated exactly one plasmid at the exact desired location.

It has thus been the objective of the present invention to address the above mentioned shortcomings of the prior art. In particular, the invention provides an easy and reliable method of plasmid copy number control. The invention also provides reliable methods for plasmid integration. And the invention provides plasmids and hosts useful in such methods.

### SUMMARY OF THE INVENTION

The invention therefore provides a method of plasmid copy number control, comprising:
i) providing a prokaryotic host comprising a plasmid, wherein the plasmid has an origin of replication activatable by a plasmid replication initiator protein (Rep), and
ii) regulating the expression of the Rep protein to adjust the plasmid copy number to a desired value.

The invention also provides a controlled replication plasmid, comprising
an origin of replication activatable by a plasmid replication initiator protein (Rep),
a rep gene coding for the Rep protein,

wherein
a) the rep gene is operably linked to a heterologous promoter and/or
b) the plasmid comprises, operably linked to a heterologous promoter, a repressor gene coding for a repressor of Rep expression.

And the invention provides a replication help dependent plasmid, comprising
an origin of replication activatable by a plasmid replication initiator protein (Rep),
optionally a promoter operably linked to a repressor gene coding for a repressor of Rep expression,

wherein the plasmid does not comprise a functional rep gene coding for the Rep protein.

The invention also provides a plasmid replication control host, comprising a controlled replication plasmid as described herein. Furthermore provided is a plasmid replication help host, comprising
i) a replication help dependent plasmid as described herein, and
ii) an expression cassette for expression of a rep gene located in the host chromosome and/or on a helper plasmid in the host.

Also provided is a cultivation method, comprising
i) providing a starter culture of a host comprising a plasmid according to the invention,
ii) cultivating said host to achieve an increase in host cell number, and
iii) during or after step ii) adjusting the plasmid copy number in the cultivated host cells.

Further provided is an integration method, comprising
i) providing, in a prokaryotic plasmid recipient host, a plasmid according to the invention comprising a selection marker,
ii) preventing replication of the plasmid while maintaining selection for the selection marker.

In particular provided is an integration method, comprising
i) providing a plasmid donor host and a plasmid recipient host, wherein
   the plasmid donor host comprises a controlled replication plasmid or a replication help dependent plasmid according to the present invention, the plasmid comprising a selectable marker, and wherein
   the plasmid recipient host does not comprise a rep gene,
ii) transferring the plasmid from the plasmid donor host to the plasmid recipient host, and
iii) simultaneously with or after step ii) preventing replication of the plasmid in the plasmid recipient host while subjecting the plasmid recipient host to selection pressure to force presence of the selectable marker in the plasmid recipient host.

### BRIEF DESCRIPTION OF THE FIGURES

FIG. 1: Schematic view of the pE194 origin of replication including regulatory genes and features. Non-coding sequences are depicted as light grey boxes, ORFs are represented as black arrows. Promoters are indicated with angled arrows. Produced proteins in their oligomeric states are depicted in dark grey. SSO: single strain origin; DSO: Double strand origin; cop gene: encoding for ‘copy control protein. Cop forms a dimer and binds a repressor to its own promoter. Transcription from Pcop promoter leads to mRNA covering cop and repF genes. repF gene: enoding for RepF protein which binds to the DSO and nicks within the DSO region as initiator of plasmid replication, ctRNA: countertranscript RNA that is transcribed from the minus strand overlapping the 5′UTR of the repF ORF, represses RepF translation initiation.

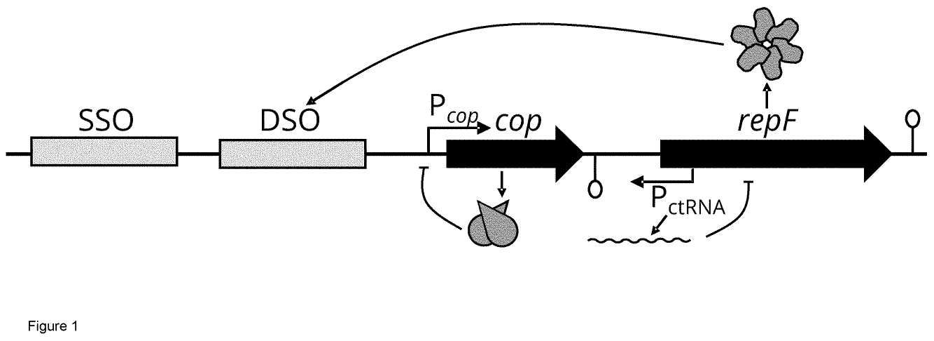

FIG. 2: A) Schematic representation of B. subtilis 168 strains carrying the indicated plasmids pKS100 (pE194 origin of replication, cop-repF) and plasmid pKS101 (pE194(ts) origin of replication; marked as cop-repF*) or plasmid pKS102 (no pE194 origin of replication) integrated into the amyE locus (Bs#073). LuxABCDE: luciferase genes; amy: amylase amyE homology region. B) B. subtilis strains were cultivated at the indicated temperatures in LB medium supplemented with erythromycin (50 μg/ml) and agitation. Samples were withdrawn after 8 h of growth and the average plasmid copy number per cell determined by quantitative real-time PCR. Relative PCN: relative plasmid copy number of indicated plasmids relative to the chromosomal terminus (per cell). Bs#073 is the integration control with one plasmid backbone per chromosomal terminus (dotted line).

FIG. 3: A) Schematic representation of B. subtilis 168 strain carrying the indicated plasmid pKS100 (pE194 origin of replication, cop-repF) and B. subtilis strain GXC1 with genomically integrated PxylA-cop expression cassette and plasmid KS100. LuxABCDE: luciferase genes; amy: amylase amyE homology region. B) Cultures of indicated strains were incubated at 37° C. in LB media containing erythromycin (50 μg/ml) without xylose (0%), with 0.125% xylose or with 0.5% xylose. Samples were withdrawn after 8 h and quantitative real-time PCR conducted to determine the average plasmid copy number per cell. The dotted line marks the copy number of one plasmid per chromosomal terminus—the ratio for integrated plasmid DNA. Relative PCN: relative plasmid copy number of indicated plasmids relative to the chromosomal terminus (per cell). PxylA: promoter of the xylA gene of B. megaterium.

FIG. 4: A) Schematic representation B. subtilis GCX1 strains with genomically integrated Pxy-lA-cop expression cassette carrying the indicated plasmids pKS100 (pE194 origin of replication, cop-repF) and plasmid pKS101 (pE194(ts) origin of replication; marked as cop-repF*). LuxA-BODE: luciferase genes; amy: amylase amyE homology region. B) The luminescence signal and cell density (OD at 600 nm) of cultures of B. subtilis GCX1 with plasmid pKS10 and plasmid pKS101 was measured after cultivation in LB media supplemented with erythromycin (50 μg/ml) at 30° C. for 8 h. The relative luminescence signal (RLU) is plotted against the different concentrations of inducer molecule xylose as indicated. The dotted line indicates the RLU of the reference control strain Bs#073 with the lux operon integrated into the genome.

FIG. 5: A) Schematic representation of B. subtilis PCX1 strains with plasmid pKS111 carrying the indicated plasmids pKS100 (pE194 origin of replication, cop-repF) and plasmid pKS101 (pE194(ts) origin of replication; marked as cop-repF*) and B. subtilis 168 strain with plasmid pKS100. LuxABCDE: luciferase genes; amy: amylase amyE homology region. B) The luminescence signal and cell density (OD at 600 nm) of cultures of B. subtilis PCX1 carrying plasmid pKS111 with plasmids pKS100 or pKS101 were measured after cultivation in LB media supplemented with erythromycin (50 μg/ml) and kanamycin (20 μg/ml) at 30° C. for 8 h. The relative luminescence signal (RLU) is plotted against the different concentrations of inducer molecule xylose as indicated. The reference strain B. subtilis 168 strain with plasmid pKS100 served as control and was cultivate without kanamycin.

FIG. 6: A) Schematic representation of B. subtilis 168 strain with plasmid pBAio-lumiBs carrying the following genetic elements: pE194 origin of replication (shown as cop-repF), an additional copy of the cop gene under the control of the PxylA promoter, LuxABCDE operon (luciferase genes under constitutive promoter Pveg) and the amylase amyE homology region (amy). B) The luminescence signal and cell density (OD at 600 nm) of the culture of B. subtilis 168 carrying plasmid pBAio-lumiBs were measured after cultivation in LB media supplemented with erythromycin (50 μg/ml) at 30° C. for 8 h. The relative luminescence signal (RLU) is plotted against the different concentrations of inducer molecule xylose as indicated. The RLU signal is lower in dependency of the concentration of inducer molecule xylose

FIG. 7: A) Schematic representation of B. licheniformis P308 strain with plasmid pBAio-lumiBI carrying the following genetic elements: pE194 origin of replication (shown as cop-repF), an additional copy of the cop gene under the control of the PxylA promoter, LuxABCDE operon (luciferase genes under constitutive promoter Pveg) and the amylase amyB homology region (amy). B. licheniformis P308 carrying pBAio lumiBI is cultivated LB media with kanamycin (20 pg/ml) and indicated xylose concentrations for 8 h at 37° C. B) Relative luminescence output RLU of plasmid pBAio-lumiBI is plotted against the indicated xylose concentration. The RLU of plasmid pBAio-lumiBI is lower upon induction of cop expression. C) Average plasmid copy number of pBAio lumiBI in B. licheniformis P308 upon cop expression with indicated xylose concentrations. Samples were withdrawn after 8 h of cultivation and the average plasmid copy number per cell determined by quantitative real-time PCR. Relative PCN: relative plasmid copy number of plasmids relative to the chromosomal terminus (per cell). The dotted line indicates the level of single copy chromosomal integration

FIG. 8: 100% Campbell recombination efficiency by combination of overexpression of cop and temperature shift. A) Schematic of the two statuses of luminescent B. licheniformis LBL cells carrying a pKS137 plasmid with a luxA homology region. The plasmid is replicating autonomously, the genome encoded luxABCDE sequence is intact and all cells show luminescence. When plasmid replication is abolished, the integration of pKS137 into the genome via homologous recombination with the homologous region (luxA′) into the luxA sequence takes place, disrupting this gene that encodes for a subunit of the luciferase. Cells with this genotype are no longer able to produce functional luciferase and show no luminescence. B) Luminescence-based evaluation of the plasmid integration rates in B. licheniformis. B. licheniformis strain LBL with plasmid pKS137 is cultivated in liquid culture during the day following plating on LB agar plates both supplemented with 20 μg/ml kanamycin. The supplementation of 0.5% inducer molecule xylose is indicated by ‘+’ and the temperature of cultivation with either 37° C. or 45° C. is indicated. Results for the following conditions are shown: no xylose in day culture and no xylose in agar plates (white bar), no xylose in day culture and 0.5% xylose in agar plates (hatched right slanted), 0.5% xylose in day culture and no xylose in agar plates (black) or 0.5% xylose in day culture and 0.5% xylose in agar plates (crosshatched).

### DETAILED DESCRIPTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50% ±10%).

As used herein, the term “gene” refers to a biochemical information which, when materialised in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

A “target gene” refers to a gene or allele having a desired property. According to the present invention the target gene preferably codes for an enzyme, and more preferably the enzyme is selected from the group consisting of amylase, catalase, cellulase, chitinase, cutinase, galactosidase, beta-galactosidase, glucoamylase, glucosidase, hemicellulase, invertase, laccase, lipase, mannanase, mannosidase, nuclease, oxidase, pectinase, phosphatase, phytase, protease, ribonuclease, transferase and xylanase, more preferably a protease, amylase or lipase, most preferably a protease.

Mutations or alterations of amino or nucleic acid sequences can be any of substitutions, deletions or insertions; the terms “mutations” or “alterations” also encompass any combination of these.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “I” symbol in the alignment indicates identical residues (which means bases for DNA or amino acids for proteins). The number of identical residues is 6.

The “−” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:

%-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138: 267-284, 1984): Tm=81.5° C.+16.6×log([Na+]{a})+0.41×%[G/C{b}]−500×[L{c}]-1−0.61×% formamide
DNA-RNA or RNA-RNA hybrids: Tm=79.8+18.5 (log10[Na+]{a})+0.58 (% G/C{b})+11.8 (% G/C{b})2−820/L{c}
oligo-DNA or oligo-RNAd hybrids:

for <20 nucleotides: Tm=2 ({In})

for 20-35 nucleotides: Tm=22+1.46 ({In})

wherein:

{a} or for other monovalent cation, but only accurate in the 0.01-0.4 M range

{b} only accurate for % GC in the 30% to 75% range

{c} L=length of duplex in base pairs

{d} Oligo, oligonucleotide

{In} effective length of primer=2×(no. of G/C)+(no. of A/T)

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1× SSC or at 42° C. in 1× SSC and 50% formamide, followed by washing at 65° C. in 0.3× SSC. Examples of medium stringency hybridisation conditions for DNA hybrids longer than 50 nucleotides encompass hybridisation at 50° C. in 4× SSC or at 40° C. in 6× SSC and 50% formamide, followed by washing at 50° C. in 2× SSC. The length of the hybrid is the anticipated length for the hybridising nucleic acid. When nucleic acids of known sequence are hybridised, the hybrid length may be determined by aligning the sequences and identifying the conserved regions described herein. 1× SSC is 0.15M NaCl and 15 mM sodium citrate; the hybridisation solution and wash solutions may additionally include 5× Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1× SSC comprising 0.1 SDS and optionally 5× Denhardt's reagent, 100 μg/ml denatured, fragmented salmon sperm DNA, 0.5% sodium pyrophosphate, followed by the washing at 65° C. in 0.3× SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic.

The term “nucleic acid construct” is synonymous with the term “expression cassette” when the nucleic acid construct contains the control sequences required for expression of a polynucleotide.

The term “control sequence” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS, shine dalgarno sequence), 3′-UTR, and transcription start and stop sites.

The term “functional linkage” or “operably linked” with respect to regulatory elements, is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. Promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule which presence in the fermentation medium is capable of affecting an increase in expression of a gene by increasing the activity of an inducer-dependent promoter operably linked to the gene. Preferably the inducer molecule is a carbohydrate or an analog thereof. In one embodiment, the inducer molecule is a secondary carbon source of the Bacillus cell. In the presence of a mixture of carbohydrates cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without be ing restricted to these. In contrast thereto, the activity of promoters that do not depend on the presence of an inducer molecule (herein called “inducer-independent promoters”) are either constitutively active or can be increased regardless of the presence of an inducer molecule that is added to the fermentation medium.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

The term “vector” is defined herein as a linear or circular DNA molecule that comprises a polynucleotide that is operably linked to one or more control sequences that provides for the expression of the polynucleotide.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single- stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques. With respect to two or more polynucleotide sequences or two or more amino acid sequences, the term “heterologous” is used to characterized that the two or more polynucleotide sequences or two or more amino acid sequences are naturally not occurring in the specific combination with each other. In particular, the term “heterologous” when referring to a promoter-gene combination means that the specific combination of promoter and gene is not found in nature. A promotor is heterologous to a gene and vice versa in particular when (a) a promoter, which in a wild type cell is operably linked to a gene A, is now operably linked instead to another gene B, or (b) where a promotor not found in nature is operably linked to a gene, or (c) where a promotor is operably linked to a gene of a sequence not found in nature.

The term “host cell”, as used herein, includes any cell type that is susceptible to transformation, transfection, transduction, conjugation, and the like with a nucleic acid construct or expression vector. Thus, the term “host cell” includes cells that have the capacity to act as a host or expression vehicle for a newly introduced DNA sequence, in particular for expression of a target gene comprised in said newly introduced DNA sequence. The host cell according to the invention is understood to be prokaryotic and preferably belongs to a genus Gram positive or Gram negative microorganisms, more preferably selected from Acetobacter, Acidithiobacillus, Aeromonas, Agrobacterium, Alcaligenes, Arthrobacter, Azotobacter, Bacillus, Campylobacter, Chromobacterium, Citrobacter, Clostridium, Comamonas, Corynebacterium, Enterococcus, Escherichia, Flavobacterium, Fusobacterium, Geobacillus, Geobacter, Gluconobacter, Helicobacter, Ilyobacter, Lactobacillus, Lactococcus, Microlunatus, Mycobacterium, Neisseria, Oceanobacillus, Paenibacillus, Pantoea, Pseudomonas, Ralstonia, Rhizobium, Rhodococcus, Saccharopolyspora, Salmonella, Serratia, Sinorhizobium, Staphylococcus, Stenotrophomonas, Streptococcus, Streptomyces, Synechocystis, Thermus, Ureaplasma, Xanthomonas and Zymonas. Among the Gram negative hosts Proteobacteria, more preferably Gammaproteobacteria, are preferred, among these in particular hosts of genus Campylobacter, Escherichia, Flavobacterium, Fusobacterium, Helicobacter, Ilyobacter, Neisseria, Pseudomonas, Salmonella, and Xanthomonas, most preferably Pseudomonas or Escherichia coli. More preferred, however, are Gram positive hosts, among those in particular those of phylum Firmicutes, more preferably any of Bacillus, Clostridium, Enterococcus, Geobacillus, Lactobacillus, Lactococcus, Oceanobacillus, Staphylococcus, Streptococcus and Streptomyces, even more preferably of order Lactobacillales or Bacillales, even more preferably of family of Bacillaceae, Paenibacillaceae or Lactobacillaceae, even more preferably of genus Lactobacillus, Paenibacillus, Oceanobacillus, Virgibacillus or Bacillus and most preferable to the any of the species Bacillus amyloliquefaciens, Bacillus clausii, Bacillus halodurans, Bacillus lentus, Bacillus licheniformis, Bacillus paralicheniformis, Bacillus pumilus, Bacillus subtilis or Bacillus velezensis, and most preferably belongs to the species Bacillus licheniformis. It is an advantage of the present invention that the host can be more or less any prokaryote in which a plasmid of the present invention is capable of reproduction, provided the presence of a functional rep gene. Thus the plasmids and hosts of the present invention are advantageously useful used in diverse fermentation processes.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell or cell line the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Methods of inducing mutations can induce mutations in random positions in the genetic material or can induce mutations in specific locations in the genetic material (i.e., can be directed mutagenesis techniques), such as by use of a genoplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucleases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well-known in the art (see reviews by Bortesi and Fischer, 2015, Biotechnology Advances 33: 41-52; and by Chen and Gao, 2014, Plant Cell Rep 33: 575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

As used herein, “wildtype” or “corresponding wildtype plant” means the typical form of an organism or its genetic material, as it normally occurs, as distinguished from e.g. mutagenized and/or recombinant forms. Similarly, by “control cell” or “wildtype host cell” is intended a cell that lacks the particular polynucleotide of the invention that are disclosed herein. The use of the term “wildtype” is not, therefore, intended to imply that a host cell lacks recombinant DNA in its genome.

As described herein, the invention provides a method of plasmid copy number control. The term “control” signifies that the average copy number per host cell of plasmids can be adjusted by a user at will. Thus, the invention allows to perform a cultivation of a host strain and changing the plasmid copy number during the cultivation. This is not possible in such cases where plasmid copy number is only self-regulated by the plasmid's wild-type replication machinery.

According to the invention a prokaryotic host is provided which comprises a plasmid. The plasmid has an origin of replication activatable by a plasmid replication initiator protein (Rep). Such Rep proteins are generally known to the skilled person. In a functional sense the Rep proteins and their corresponding wild-type mechanisms of plasmid copy number control can be categorized into two groups: In the first and preferred group, the Rep protein effects plasmid replication, typically by binding to the origin of replication, in any physiologically acceptable concentration of the Rep protein. Such plasmids, origins of replication, Rep proteins and copy number control products (Cop and/or antisense RNA, also further described herein) are described in detail in Khan, Microbiology and Molecular Biology reviews, 1997, 442-455; the contents of this document is incorporated herein in its entirety. Well known plasmids are those belonging to the family of pBR322, pUC19, pACYC177 and pACYC184, permitting replication in E. coli, and pUB110, pE194, pTA1060, and rhoAMbetal, permitting replication in Bacillus. Typical plasmids falling into the first group as described by Khan belong to the families of pLS1 or pUB110. In the second group, the Rep protein acts as its own repressor when expressed in high concentration. Such Rep proteins and their mechanism of plasmid copy number autoregulation are described in lshiai et al., Proc. Natl. Acad. Sci USA, 1994, 3839-3843, and Giraldo et al., Nature Structural Biology 2003, 565-571. Unless specifically indicated herein or as long as technically sensible, plasmid copy number control according to the present invention is effected by either the first group or the second group of copy number regulation mechanisms. However, preference is given to the first group.

According to the invention, the expression of the Rep protein is regulated to adjust the plasmid copy number to a desired value. Thus, by interference with the host cell it is possible to increase or decrease the average plasmid copy number at will. In particular, by direct and specific interference with the expression of the Rep protein the invention thus allows to modify the plasmid copy number without having to subject the host to physiologically challenging conditions like extreme temperature shifts. It is a particular advantage of the invention that the plasmid copy number can be adjusted by the user at substantially any time and is not finally determined by the incorporation of a selected origin of replication (as described in WO2007035323), a special promoter for expression of the rep gene or the like.

Expression of the Rep protein is preferably regulated by one or more of

a) increasing expression of a rep gene coding for the Rep protein,

b) decreasing expression of a rep gene coding for the Rep protein,

c) decreasing expression of a repressor gene coding for a repressor of Rep expression,

d) increasing expression of a repressor gene coding for a repressor of Rep expression.

The expression of the Rep protein can be regulated by modifying the transcription rate of a gene (rep) coding for the Rep protein and/or by modifying the translation rate of mRNA coding for the Rep protein. Thus, expression of the Rep protein can be increased by increasing the transcription and/or translation rate of the rep gene and its respective mRNA. Correspondingly, decrease of Rep protein expression can be effected by decreasing the transcription and/or translation rate of the rep gene and its respective mRNA.

An increase of gene expression by increasing transcription efficiency according to the invention is preferably achieved by putting the respective gene under control of a heterologous inducible promoter. Such promoters are known to the skilled person for any host microorganism, in particular for those specified herein. Typically the transcription rate of such promoters is regulated by one or more transcription factors responsive to an external stimulus, preferably addition of a substance to the host's medium. It is an advantage of the present invention that the method of plasmid copy number control can be performed by employing such well-tested and reliable promoters and inductors. Suitable inductors and corresponding promoters well known to the skilled person are IPTG, lactose, xylose, tetracycline and derivates thereof, e.g. doxocycline.

An inducible promoter, also called inducer dependent promoter, is understood herein as a promoter that is increased in its activity to enable transcription of the gene to which the promoter is operably linked upon addition of an “inducer substance” to the cultivation or fermentation medium. Thus, for an inducer-dependent promoter the presence of the inducer molecule triggers via signal transduction an increase in expression of the gene operably linked to the promoter. The gene expression prior activation by the presence of the inducer molecule does not need to be absent, but can also be present at a low level of basal gene expression that is increased after addition of the inducer molecule. The “inducer molecule” is a molecule which presence in the fermentation medium is capable of affecting an increase in expression of a gene by increasing the activity of an inducer-dependent promoter operably linked to the gene. Preferably the inducer molecule is a carbohydrate or an analog thereof. In one embodiment, the inducer molecule is a secondary carbon source of the Bacillus cell. In the presence of a mixture of carbohydrates cells selectively take up the carbon source that provide them with the most energy and growth advantage (primary carbon source). Simultaneously, they repress the various functions involved in the catabolism and uptake of the less preferred carbon sources (secondary carbon source). Typically, a primary carbon source for Bacillus is glucose and various other sugars and sugar derivates being used by Bacillus as secondary carbon sources. Secondary carbon sources include e.g. mannose or lactose without being restricted to these.

Well known examples of inducible promoters are the PBAD promoter from E. coli regulated by the araC that alters its conformation and binds as dimer to the operator sites I₁ and I₂. upon addition of arabinose, and the mannose-inducible promoter system PmanP from Bacillus regulated by the activator manR. Inducible promoters such as lacUV5 promoter, the T7-phage promoter for expression in E. coli and the Pspac-I and Ppac-I promoters in Bacillus are negatively regulated by the lac repressor (encoded by lacI gene) binding in the absence of an inducer molecule to its specific lac operator sites either within the promoter sequences, e.g. between the −35 and −10 sigA recognition sites, or vicinity, i.e 3′ or 5′ of the promoter sequence to prevent transcription. Another example is the PxylA inducible promoter system from Bacillus megaterium widely used for Bacillus expression systems. The PxylA promoter is negatively regulated by the xylR repressor protein binding comprising the xylR operator sites 3′ of the transcriptional start site. Synthetic inducible expression systems with the theophylline riboswitch (Topp S, Reynoso C M, Seeliger J C, et al. Synthetic riboswitches that induce gene expression in diverse bacterial species; Appl Environ Microbiol. 2010;76(23):7881-7884) applicable in a broad range of different bacterial species have been developed.

Examples of inducer dependent promoters are given in the table below by reference to the respective operon:

| Operon | Regulator a) | Type b) | Inducer | Organism |
|---|---|---|---|---|
| sacPA | SacT | AT | sucrose | B. subtilis |
| sacB | SacY | AT | sucrose | B. subtilis |
| bgl PH | LicT | AT | β-glucosides | B. subtilis |
| licBCAH | LicR | A | oligo-β-glucosides | B. subtilis |
| levDEFG | LevR | A | fructose | B. subtilis |
| sacL | | | | |
| mtlAD | MtlR | A | mannitol | B. subtilis |
| manPA-yjdF | ManR | A | mannose | B. subtilis |
| manR | ManR | A | mannose | B. subtilis |
| bglFB bglG | BglG | AT | β-glucosides | E. coli |
| lacTEGF | LacT | AT | lactose | L. casei |
| lacZYA | lacI | R | Allolactose; IPTG | E. coli |
| | | | (Isopropyl β-D-1- | |
| | | | thiogalactopyranoside) | |
| araBAD | araC | AR | L-arabinose | E. coli |
| tetAR | TetR | R | Tetracycline/ | E. coli |
| | | | anhydrotetracycline/ | |
| | | | doxycycline | |
| xylAB | XylR | R | Xylose | B. subtilis |

a): transcriptional regulator protein
b): A: activator
AT: antiterminator
R: repressor
AR: activator/repressor

Correspondingly a decrease of gene expression by decreasing transcription efficiency according to the invention is preferably achieved by putting the respective gene under control of a heterologous silencable promoter. Such promoters are known to the skilled person for any host microorganism, in particular for those specified herein. Typically the transcription rate of such promoters is regulated by one or more transcription factors responsive to an external stimulus, preferably addition of a substance to the host's medium. It is an advantage of the present invention that the method of plasmid copy number control can be performed by employing such well-tested and reliable promoters and silencer substance. Suitable silencer substances and corresponding promoters well known to the skilled person are tet-off promoter systems applying tetracycline and derivates thereof, e.g. doxocycline, riboswitch promoter systems not limited to coenzyme B12 riboswitch, lysine riboswitch, TPP riboswitch (Winkler W C, Breaker R R. Regulation of bacterial gene expression by riboswitches. Annu Rev Microbiol. 2005; 59:487-517). Thus, the rep gene according to the invention can be operably linked to a heterologous inducible promoter in an expression cassette. The rep gene under control of an inducible promoter can be present on the plasmid and/or in the host's chromosome. In both cases the transcription rate of the rep gene and correspondingly the expression of the Rep protein is maximal when the corresponding inductor is provided to the host strain, in the absence of such inductor the transcription rate and thus the expression of the Rep protein is decreased. Thus, the expression of the Rep protein can be temporarily increased by addition of the inductor for a limited time, for example during a fermentation process. This is a particular advantage of the present invention. For example it allows to resurrect, in the absence of the inductor, a host strain culture from cryoconservation and to grow a starter culture which comprises only a low plasmid copy number. As indicated above microorganisms are metabolically challenged when they experience a change of medium or other environmental conditions. By not forcing the host to maintain a high plasmid copy number under such conditions the invention allows to increase the speed of host adaptation to the change of growth conditions, thereby allowing for a faster onset of exponential growth. It is a further advantage of the present invention that even under metabolic tress conditions a selection pressure can still be maintained, thereby preventing plasmid loss from the host culture.

Increasing the expression of the Rep protein leads to an increase of plasmid copy number in those cases where the Rep protein belongs to the first and preferred group of Rep proteins. In the second group, increasing the expression of the Rep protein only leads to an increase in plasmid copy number in a Rep protein specific concentration range. For such plasmid copy number regulation systems, absence of the Rep protein leads to a loss of plasmid from the host due to a lack of plasmid replication initiators. A high concentration of Rep protein leads to Rep autoinactivation, typically by dimerization of the Rep protein. Thus, for the second group the invention still allows to temporarily increase the plasmid copy number by increasing Rep expression from zero to an optimal Rep concentration, but also allows to temporarily reduce the plasmid copy number by increasing Rep concentration beyond the optimal Rep concentration, thereby forcing Rep into autorepression.

The rep gene may also be operably linked to a heterologous silencable promoter in an expression cassette. Again, the rep gene under control of a silencable promoter can be present on the plasmid and/or in the host's chromosome. In both cases the addition of a silencer substance reduces the transcription rate of the rep gene. Correspondingly the expression of the Rep protein is maximal in the absence of the silencer substance and is reduced in the presence of the silencer substance. For Rep proteins of the first group such configuration is advantageous when a high plasmid copy number is desired during most stages of host cultivation and a low copy number is only desired during specific stages, e.g. at a change of medium, during resurrection after cryoconservation or in the earliest stages of fermenter inoculation. By only having to add the silencer substance during such exceptional stages of low desired plasmid copy number the invention allows to perform all other handling of the host at higher plasmid copy numbers without having to add the silencer substance. For Rep proteins of the second group such configuration is advantageous to temporarily reduce Rep concentration from autorepression levels to plasmid replication permissive levels.

Expression of the Rep protein can, in addition or alternatively to coupling to an inducible or silencable promoter, also be modified by the expression of a repressor of rep gene expression under control of a heterologous promoter. Such repressor can be a nucleic acid or a protein. In plasmids of pLS1 family, for example, both types of repressors are natively employed to regulate plasmid copy number, i.e. a copA antisense RNA hybridizing to Rep-coding mRNA, and a CopB repressor protein of rep gene expression. Employing a native repressor of rep gene expression has the additional advantage that at least one copy of the rep gene can remain under control of the native promoter of Rep expression.

Preferably at least one rep gene is operably linked to a native rep gene promoter and the repressor is a repressor of the native rep gene promoter. Thus it is possible for example to provide an unmodified high copy number plasmid in a host which comprises, in its chromosome or on a helper plasmid, an expression cassette comprising a copy of the repressor gene under control of a heterologous inducible or silencable promoter. It is an advantage of such plasmid-host-systems that the host strain only needs to be genetically modified once by introducing the repressor expression cassette; the resulting host can then be used to control the copy number of any plasmid of the compatible rep gene promoter by expressing the repressor in the desired concentration at each point in time to obtain the desired level of Rep expression and, in turn, the desired plasmid copy number.

The invention also provides, in addition or as an alternative to an inducible or silencable promoter, the use of a heterologous constitutive promoter. This results in amounts of repressor that lead to a constant copy number in that cell. The expression rate of constitutive promoters can be tuned according the desired expression level as described, for example, by Guiziou et al, (2016): Nucleic Acids Res. 44(15), 7495-7508 or anti-sigma factors as described in WO2013148321.

The repressor gene can be under control of a heterologous inducible promoter. In such cases, addition of the inductor to the host results in an increase of repressor gene expression and consequently to a reduction of Rep protein expression as described above. Where a repressor gene is under control of a heterologous silencable promoter, addition of the silencer substance to the host results in a decrease of repressor gene expression and consequently to an increase of Rep protein expression as described above.

Preferably rep gene expression is under control of both a repressor nucleic acid and of a repressor protein (herein also called “Cop protein”). In such systems one first gene coding for a first repressor is preferably located on the plasmid and the expression cassette of the other gene coding for the second repressor is preferably located anywhere, preferably integrated into the host's chromosome. Thus the first repressor functions as an autoregulator of plasmid copy number: When the plasmid copy number increases, the gene dosage of the first repressor correspondingly also increases. This, in turn, leads to an increase in first repressor concentration and thus to a repression of rep gene expression, thereby limiting the maximum plasmid copy number without requiring interference by the user. When the plasmid copy number decreases, the gene dosage of the first repressor correspondingly also decreases. This, in turn, leads to a reduction of first repressor concentration and thus to an increase in rep gene expression, thereby preventing, again without need for an action of the user, plasmid loss or dropping of plasmid copy number below a minimum threshold. When rep gene expression is under control of both a repressor nucleic acid and of a repressor protein it is preferred that the first repressor is a repressor nucleic acid, most preferably an antisense RNA, and the expression cassette comprising a promoter and, operably linked thereto, the first repressor gene is preferably located on the plasmid whose copy number is to be controlled. Such configuration is particularly advantageous because in this way autoregulation does not depend on metabolically costly production of a repressor protein; instead, the transcript of the first repressor gene is sufficient to repress Rep expression in the desired intensity. A further advantage is that nucleic acid repressors are faster to produce than protein repressors and generally have a shorter half-life time, they can thus be used to control Rep expression with only a very short delay. And nucleic acid repressors, in the form of antisense nucleic acids, are independent of the sequence of the promoter that drives rep gene expression. Thus, a nucleic acid repressor can repress Rep expression by preventing or reducing translation of a rep mRNA transcript even if the rep gene is under control of a heterologous promoter.

Correspondingly the invention also provides plasmids adapted to plasmid copy number control. In particular the invention provides a controlled replication plasmid comprising an origin of replication activatable by a plasmid replication initiator protein (Rep) and a rep gene coding for the Rep protein, wherein the rep gene is operably linked to a heterologous promoter, and wherein the promoter preferably is inducible or silencable. As described above such plasmids allow to increase or decrease Rep protein expression by addition of an inductor or silencer substance. When the rep gene is capable of being repressed by at least one repressor, then this repressor is preferably not provided by the plasmid but by the host, preferably by a repressor expression cassette integrated into the host's chromosome and/or a repressor expression cassette located on a helper plasmid. As indicated above, the invention also provides a plasmid comprising a rep gene under control of a heterologous, preferably inducible or silencable promoter, and also comprising a gene coding for a nucleic acid repressor of Rep expression. Such antisense nucleic acid repressors are independent of the promoter that drives expression of the rep gene.

The invention also provides a controlled replication plasmid comprising an origin of replication activatable by a plasmid replication initiator protein (Rep) and a rep gene coding for the Rep protein, wherein the the plasmid comprises, operably linked to a heterologous promoter, a repressor gene coding for a repressor of Rep expression. Again, the heterologous promoter preferably is inducible or silencable. When the repressor is a repressor protein, then the rep gene is under control of a promoter repressible by the repressor protein. In such cases the rep gene is preferably operably linked to a native rep gene promoter or to a promoter derived from a native rep gene promoter, wherein the derived promoter still allows binding of the repressor protein. It is a particular advantage of a plasmid comprising an origin of replication activatable by a plasmid replication initiator protein (Rep), a rep gene coding for the Rep protein and, under control of a heterologous inducible or silencable promoter, a repressor gene coding for a repressor of Rep expression that the copy number of such plasmid can be controlled by application of the respective inducer or silencer substance without any modification to the host's genetic material. Thus such all-in-one plasmids are particularly versatile. Particularly preferred uses of such plasmids are described herein, in particular in the figures and examples.

Similarly the invention provides a controlled replication plasmid comprising an origin of replication activatable by a plasmid replication initiator protein (Rep) and a rep gene coding for the Rep protein, wherein the rep gene is operably linked to a heterologous promoter, and further comprises, operably linked to a heterologous promoter, a repressor gene coding for a repressor of Rep expression. Preferably at least the heterologous promoter operably linked to the repressor gene is inducible or silencable. One such plasmid has been described above, i.e. a plasmid comprising a nucleic acid repressor to repress the rep gene that is under control of a heterologous promoter.

Furthermore the invention provides a replication help dependent plasmid, comprising
an origin of replication activatable by a plasmid replication initiator protein (Rep), and
optionally a promoter operably linked to a repressor gene coding for a repressor of Rep expression,

wherein the plasmid does not comprise a functional rep gene coding for the Rep protein.

As described herein a replication help dependent plasmid still requires the activity of a Rep protein. However, the Rep protein is not provided in cis by a corresponding expression cassette located on the plasmid. Instead, the Rep protein is provided in trans by a corresponding expression cassette located in the host's chromosome and/or on a helper plasmid. Such helper plasmid preferably does not depend on the activity of the Rep protein for replication and thus is replicated independently of the replication help dependent plasmid. Providing the rep gene in trans has the advantage that the corresponding rep gene expression cassette only needs to be established once in the host, preferably in its chromosome, and is thus available for any replication help dependent plasmid or any other controlled replication plasmid introduced into the host. A replication help dependent plasmid has particular advantages. First of all it is replicating only in a competent host. Thus, a replication help dependent plasmid is a particularly safe vector which inherently prevents accidental transformation of hosts not expressing the Rep protein. Furthermore replication help dependent plasmids particularly facilitate plasmid integration methods as described herein.

The replication help dependent plasmid does not comprise a functional rep gene. Absence of a functional rep gene can be achieved by excision of a rep gene from a wild-type plasmid comprising a rep gene. Furthermore, the rep gene can be inactivated, for example by introduction of one or more premature stop codons or by other amino acid changes to code for a non-functional protein. In addition the gene, preferably coding for a non-functional protein, can be devoid of a functional promoter. Most preferably the replication help dependent plasmid does not comprise a nucleic acid sequence having at least 95%, more preferably at least 90%, even more preferably at least 80% sequence identity to a rep gene coding for any of the following amino acids as identified by their Uniprot identifiers according to table 1.

The replication help dependent plasmid preferably further comprises a gene coding for a repressor which, when expressed in a host comprising an expression cassette for expression of the Rep protein, represses expression of the Rep protein. As described above such host provides the rep gene in trans. By controlling expression of the rep gene provided by the host via the repressor the invention allows to control plasmid copy number. In such cases it is particularly advantageous to have a rep gene on the replication help dependent plasmid coding for a nucleic acid repressor of Rep protein expression. As described above, nucleic acid repressors are particularly useful for example to establish a feedback mechanism to limit the maximum plasmid copy number without requiring user interference.

Particularly preferred features applicable in controlled replication and in replication help dependent plasmids are described hereinafter:

As described above a heterologous promoter operably linked to a repressor and/or rep gene is responsive to a change of an external regulator concentration or a metabolite concentration. Heterologous promoters responsive to external regulator concentrations have been described herein as inducible or silencable promoters. A heterologous promoter responsive to a change of a metabolite concentration allows to automatically induce or silence the gene operably linked to the promoter under predefined metabolic conditions. It is particularly advantageous to provide, on the plasmid of the present invention, in the host's chromosome and/or on a helper plasmid, a repressor gene under control of a promoter such that the repressor gene is silenced during late stages of a fermentation, when host cells cease to grow exponentially. At such stages close to the end of a batch fermentation host viability is no longer of concern. Instead, maximising “last minute” expression of a target gene located on the plasmid may be more desirable. Such “last minute” induction of expression can be achieved by removing repression of plasmid copy number control, thereby increasing plasmid copy number and thus target gene dosage. Suitable promoters responsive to such end of fermentation conditions are, for example, promoters silenced under severe limitation or those silenced at the onset of sporulation. An example of such promoters is abrB of Bacillus subtilis.

The plasmid according to the present invention preferably is a rolling circle replication type plasmid as described above. Such plasmids allow for a particularly easy and reliable control of replication because the Rep protein does not act an autorepressor. Thus, changing the concentration of the Rep protein in a host cell does not have to be as fine-tuned as for second Rep group plasmids.

Furthermore the plasmid preferably comprises one or more of
a selectable marker,
a target gene,
an expression cassette comprising a promoter operably linked to a target gene,
a recombination site.

Selectable markers allow to secure presence of the plasmid in the host cell. As described above selectable markers also allow to secure highly efficient integration of the plasmid of the present invention into a foreing nucleic acid by homologous recombination, preferably into the host's chromosome.

The target gene preferably codes for an enzyme, and more preferably the enzyme is selected from the group consisting of amylase, catalase, cellulase, chitinase, cutinase, galactosidase, beta-galactosidase, glucoamylase, glucosidase, hemicellulase, invertase, laccase, lipase, mannanase, mannosidase, nuclease, oxidase, pectinase, phosphatase, phytase, protease, ribonuclease, transferase and xylanase, more preferably a protease, amylase or lipase, most preferably a protease. It is a particular advantage of the present invention that the plasmid of the present invention is useful for expression of such a variety of target genes common in industrial fermentation processes.

The invention also provides a plasmid replication control host, comprising a controlled replication plasmid as described herein. Such host is particularly adapted to a change in plasmid copy number according to the needs defined by a user. In particular such host allows to temporarily decrease the plasmid copy number when preparing a cryoconservation culture or when resurrecting a cryoconservation culture sample.

The plasmid replication control host preferably provides, in trans, a repressor gene coding for a repressor of Rep expression operably linked to a heterologous inducible or silencable promoter. This allows to temporarily reduce the plasmid copy number by inducing expression of the rep gene provided by the host or to temporarily increase plasmid copy number by reducing expression of the rep gene provided by the host. Preferably the plasmid replication control host provides a gene, in trans, coding for a repressor protein of Rep expression as described herein.

The invention also provides a plasmid replication help host, comprising
i) a replication help dependent plasmid according to the present invention, and
ii) an expression cassette for expression of a rep gene located in the host chromosome and/or on a helper plasmid in the host.

As described above such plasmid replication help host provides the Rep protein in trans and is thus particularly suitable for maintaining a replication help dependent plasmid. As such, the plasmid replication help host is part of a security framework to prevent accidental transmission of the replication help dependent plasmid to another strain which does not provide the Rep protein in trans.

In view of the above advantages the invention also provides a cultivation method, comprising
i) providing a starter culture of a host comprising a plasmid according to the present invention,
ii) cultivating said host to achieve an increase in host cell number, and
iii) during or after step ii) adjusting the plasmid copy number in the cultivated host cells.

As described herein the plasmid can be a controlled replication plasmid or a replication help dependent plasmid. In the latter case the host should be a plasmid replication help host.

In the above cultivation method according to the present invention, a starter culture is provided. Such starter culture preferably is a culture that had been maintained under significantly different metabolic conditions than those experienced in step ii) of the cultivation method. Preferably the starter culture is a culture sample to be resurrected from cryoconservation. Also preferably the starter culture is a culture sample for inoculation of a fermenter and is thus subject to a change of cultivation volume from a starter cultivation volume of up to 100 ml to a fermentation volume of at least 1 cubic meter.

In step ii) the starter culture is cultivated to increase the host cell number. Typically this results in an increase in optical density of the culture medium. The invention is not restricted to a particular medium or specific method to achieve the increase in host cell number. Instead the cultivation method of the present invention is advantageously broadly applicable in industrial fermentation processes.

During or, less preferred, after step ii) the plasmid copy number in the cultivated host cells is adjusted. The adjustment preferably is a temporary reduction of plasmid copy number, an increase of plasmid copy number or a reduction followed by an increase of copy number.

As described above the cultivation method as provided herein allows to multiply a host even under metabolically challenging conditions, for example at a change of medium or at resurrection after cryoconservation, by maintaining a low copy number during such conditions and only later increasing the plasmid copy number when it can be expected that the host is capable of sustaining the high plasmid copy number without compromising growth.

Adjustment of plasmid copy number can be delayed after start of step ii). Typically a starter culture is placed in or on a new medium to initiate host cell multiplication. Reduction of plasmid copy number is preferably done at the start of step ii). This way the host cell culture is relieved from the metabolic burden of high plasmid copy number maintenance. Increase of plasmid copy number preferably starts when the host cell number has increased by at least 25%, more preferably by at least 33%, even more preferably by at least 45%, even more preferably by at least 50%, even more preferably by at least 75% and even more preferably by at least 90%. Under these conditions the share of host cells which have successfully adapted to the new cultivation conditions is high enough to shoulder the additional metabolic burden of increased plasmid replication.

The invention correspondingly also provides a starter culture preparation method, wherein the plasmid copy number in a host culture is adjusted, preferably reduced, while the host cells of the culture are allowed to multiply. This way the invention provides a particularly advantageous method to complement the above starter culture cultivation method. By reducing the plasmid copy number in a host cell culture before cryoconservation, medium change, inoculation of a large volume of a new medium or other significant metabolically stressing events the host cells are relieved from some of the stress of plasmid multiplication. By using this starter culture preparation method the invention advantageously provides starter cultures with already reduced plasmid copy number. Such starter cultures are thus, even without any user interaction, relieved of the initial metabolic burden of maintaining a high plasmid copy number immediately after start of a cultivation of these starter cultures, for example after resurrection of such cryoconserved starter culture. The starter culture preparation method thus advantageously provides a starter culture wherein, in step ii) of the above cultivation method, no additional reduction of plasmid copy number is required.

Furthermore the invention provides a starter culture comprising, in a starter medium, a plasmid replication control host or plasmid replication help host according to the present invention, wherein the plasmid copy number of the host increases after transfer of the host into LB medium, i.e. a medium of 10 g tryptone, 10 g NaCl, 5 g yeast extract for 950 g water. Thus such starter culture of the invention advantageously achieves a natural increase in plasmid copy number even without requiring additional user interference. It is to be understood that the starter culture can be added to any suitable medium chosen in view of the host's metabolic requirements and is not limited to growth in LB media.

According to the invention a low plasmid copy number preferably is at most half, more preferably at most 35%, more preferably at most 30%, more preferably at most 25%, more preferably at most 20%, more preferably at most 10% of the maximum plasmid copy number, wherein the maximum plasmid copy number is determined as the maximum of

a) the equilibrium plasmid copy number of the corresponding wild type plasmid in the corresponding host and

b) the maximum plasmid copy number of the plasmid of the present invention in the corresponding host achieved by, as applicable, increase of Rep expression by application of an inductor or silencer substance.

Preferably, a low plasmid copy number is 1-15 and a high plasmid copy number is 20-100. More preferably a low plasmid copy number is 1-10 and a high plasmid copy number is 30-60. For example for plasmids of the pE194 replicon (pLS1 family) the preferred high plasmid copy number is 30-40 plasmids per host cell and the preferred low plasmid copy number is 1-10 plasmids per host cell.

Provided herein is also an integration method. As described above integration according to the invention can be into a host's chromosome but also into a further plasmid. However, according to the invention it is preferred that the host used in the integration method does not comprise a plasmid other than a plasmid of the present invention. This way the integration method is not prone do replication incompatibilities of two different plasmids present in the host.

In step i) a prokaryotic plasmid recipient host is provided comprising a plasmid of the present invention. The plasmid preferably is a controlled replication plasmid and thus comprises an expression cassette for rep gene expression. The plasmid can also be a replication help dependent plasmid. The plasmid used in the integration method according to the present invention comprises a selection marker. The selection marker allows to ascertain the presence of at least the selection marker nucleic acid segment in the recipient host.

In step ii) replication of the plasmid is prevented while maintaining selection for the selection marker. The ongoing selection pressure enforces the maintenance of at least the selection marker nucleic acid segment in the recipient host. However, as the plasmid is incapable of replication during step ii), the plasmid—in the absence of the selection pressure—would be lost from the recipient host. Thus the continued presence of the plasmid is of utmost importance for survival of the recipient host. In such situation the recipient host is forced to incorporate the plasmid into the host chromosome, because the host is only capable to withstand ongoing selection pressure after incorporation of the plasmid.

Prevention of plasmid replication according to the invention can be effected by one or more of the following methods:
withdrawing a required inductor substance when the rep gene is operably linked to an inducible heterologous promoter,
addition of a silencer substance when the rep gene is operably linked to a silencable heterologous promoter,
production of a repressor of Rep expression by addition of an inductor substance when the repressor gene is operably linked to an inducible promoter,
production of a repressor of Rep expression by withdrawal of a silencer substance when the repressor gene is operably linked to a silencable promoter.

Preferably the repressor is a protein repressor and the rep gene is under control of a promoter repressible by the protein repressor. It is a particular advantage that the integration method of the present invention can be applied both to generally used plasmids without modification of the plasmid's replication operon. In this case the recipient host provides the represssor gene in trans under control of an inducible or silencable promoter. However, the integration method is also applicable without the host having to provide the repressor gene in trans. In this case the plasmid provides a repressor gene under control of an inducible or silencable promoter. Of course, both the plasmid and the host can provide the prepressor gene under control of an inducible or silencable promoter, thereby advantageously increasing repressor concentration in the host during step ii) of the integration method.

As shown in the examples it is a particular advantage that the present invention achieves, with high reliablility, complete plasmid integration into the host's chromosome.

In a particular integration method according to the present invention, the plasmid is transferred from a plasmid donor host to the plasmid recipient host. This method advantageously allows to cultivate the plasmid in a plasmid donor host, for example with a high plasmid copy number, and then to transfer the plasmid to the recipient host in which it is not capable of replication. Transfer of plasmid can be effected by any suitable means, for example by electroporation or by conjugation. Conjugation is the particularly preferred method of transfer because it is very gentle to the recipient host. This is of particular importance because of the selection pressure applied to the recipient host. Under such selection pressure the recipient host is under severe stress to immediately start expression of the plasmid's selection marker. Recipient host survival under such conditions is significantly impaired when the host also has to recover from the effects of electroporation. The inventors have observed that after electroporation recipient host cells just die, thereby reducing effectiveness of an antibiotic added to the recipient host cell culture. This, in turn, leads to a reduction of selection pressure and thus reduces the need of the recipient host to integrate the plasmid nucleic acid. Furthermore many hosts with interesting properties in industrial fermentation processes cannot reliably electroporated.

The invention thus provides an integration method comprising the steps:
i) providing a plasmid donor host and a plasmid recipient host, where
   the plasmid donor host comprises a controlled replication plasmid or a replication help dependent plasmid according to present invention, the plasmid comprising a selectable marker, and
   the plasmid recipient host does not comprise a rep gene,
ii) transferring the plasmid from the plasmid donor host to the plasmid recipient host, and
iii) simultaneously with or after step ii), preventing replication of the plasmid in the plasmid recipient host while subjecting the plasmid recipient host to selection pressure to force presence of the selectable marker in the plasmid recipient host.

As described above transfer of the plasmid preferably is effected by conjugation. The aforementioned three-step integration method is particularly advantageous because the absence of any Rep gene in the recipient host puts a very high pressure on the host to integrate the plasmid into the chromosome because this is the only way of maintaining presence of the selection marker nucleic acid sequence. Without integration the selection marker nucleic acid sequence would be lost in daughter cells, which, in turn would be incapable of growth.

The plasmid preferably comprises a recombination site to facilitate recombination with a compatible site of a nucleic acid of the replication recipient host. By offering a recombination site on the plasmid one event of homologous recombination between the plasmid and the host nucleic acid, preferably the host chromosome, is sufficient to achieve complete integration of the plasmid. This is a particular advantage because it guarantees that all or substantially all surviving recipient host cells will have integrated the complete plasmid. Thus, the integration method of the present invention is not only efficient in producing recombinant recipient hosts but is also reliable.

The invention is hereinafter further explained by the examples and figures. The examples and figures are offered as means of further illustration of selected aspects in the context of the present invention and not meant to limit the understanding of the present invention or the scope of the claims.

### EXAMPLES

Unless otherwise stated the following experiments have been performed by applying standard equipment, methods, chemicals, and biochemicals as used in genetic engineering and fermentative production of chemical compounds by cultivation of microorganisms. See also Sambrook et al. (Sambrook, J. and Russell, D. W. Molecular cloning. A laboratory manual, 3rd ed, Cold Spring Harbor Laboratory Press, Cold Spring Harbor, NY. 2001) and Chmiel et al. (Bioprocesstechnik 1. Einführung in die Bioverfahrenstechnik, Gustav Fischer Verlag, Stuttgart, 1991). Standard methods in molecular biology not limited to cultivation of E.coli microorganisms, electroporation of DNA, isolation of genomic and plasmid DNA, PCR reactions, cloning technologies were performed as essentially described by Sambrook and Rusell. (Sambrook, J. and Russell, D. W. Molecular cloning. A laboratory manual, 3rd ed, Cold Spring Harbor Laboratory Press, Cold Spring Harbor, NY 2001.)

TABLE 1
sources of plasmids and corresponding rep and repressor
genes in NCBI (accession date Feb. 6, 2020)

| Plasmid | Accession | cop | cooordinates | rep | cooordinates | |
|---|---|---|---|---|---|---|
| Name | number | gene | nt | gene | nt | comment |
| pMV158 | X15669.1 | repA | 655-792 | repB | 853-1485 | |
| pLS1 | M29725.1 | repA | 655-792 | repB | 853-1485 | |
| pFX2 | X54310.1 | copX | 1341-1502 | repX | 1569-2267 | |
| pA1 | Z11717.1 | repA | 969-1112 | repB | 1188-1778 | |
| pE194 | V01278.1 | cop | 1011-1178 | repF | 1259-1858 | cop gene (Kwak et |
| | | | | | | al., 1994, Journal of |
| | | | | | | Bacteriology, p- 5044- |
| | | | | | | 5051); repF gene |
| | | | | | | (Villafane et al., |
| | | | | | | 1989, J. Bacteriol. |
| | | | | | | 171 (5), 2866-2869 - |
| | | | | | | rep geme) - |
| | | | | | | accession number |
| | | | | | | M17811.1 |
| pSMQ172 | AF295100.1 | cop | 262-399 | rep | 460-1131 | |
| pLA106 | D88438 | repA | 1893-2048 | repB | 2105-2686 | |
| pLC2 | AJ518839 | ORF1 | 269-439 | rep | 523-1188 | |
| pLF14 | AJ518839 | repA | 1642-1800 | repB | 1881-2570 | |
| pWV01 | X56954.1 | ORFC | 585-746 | repA | 813-1511 | |
| pSH71 | A09339.1 | | 964-1116 | | 1279-1887 | |

Electrocompetent Bacillus licheniformis cells and electroporation Transformation of DNA into B. licheniformis strain DSM 641 and ATCC 53926 is performed via electroporation. Preparation of electrocompetent B. licheniformis cells and transformation of DNA is performed as essentially described by Brigidi et al (Brigidi, P., Mateuzzi, D. (1991). Biotechnol. Techniques 5, 5) with the following modification: Upon transformation of DNA, cells are recovered in 1 ml LBSPG buffer and incubated for 60 min at 37° C. (Vehmaanperä J., 1989, FEMS Microbio. Lett., 61: 165-170) following plating on selective LB-agar plates.

In order to overcome the B. licheniformis specific restriction modification system of B. licheniformis strains DSM 641 and ATCC 53926 plasmid DNA is isolated from Ec#098 cells as described below. For transfer in Bacillus lichenformis restrictase knockout strains, plasmid DNA is isolated from E. coli INV110 cells (Life technologies).

Electrocompetent Bacillus pumilus Cells and Electroporation

Transformation of DNA into B. pumilus DSM14395 is performed via electroporation. Preparation of electrocompetent B. pumilus DSM14395 cells and transformation of DNA is performed as described for Bacillus licheniformis cells.

In order to overcome the Bacillus pumilus specific restriction modification system plasmid DNA is isolated from E. coli DH10B cells and plasmid DNA is in vitro methylated with whole cell extracts from B. pumilus DSM14395 according to the method as described for B. licheniformis in patent DE4005025.

Plasmid Isolation

Plasmid DNA was isolated from Bacillus and E. coli cells by standard molecular biology methods described in (Sambrook, J. and Russell, D. W. Molecular cloning. A laboratory manual, 3rd ed, Cold Spring Harbor Laboratory Press, Cold Spring Harbor, NY. 2001) or the alkaline lysis method (Birnboim, H. C., Doly, J. (1979). Nucleic Acids Res 7(6): 1513-1523). Bacillus cells were in comparison to E. coli treated with 10 mg/ml lysozyme for 30 min at 37C prior to cell lysis.

Annealing of Oligonucleotides to Form Oligonucleotide-Duplexes

Oligonucleotides were adjusted to a concentration of 100 μM in water. 5 μl of the forward and 5 μl of the corresponding reverse oligonucleotide were added to 90 μl 30 mM Hepes-buffer (pH 7.8). The reaction mixture was heated to 95° C. for 5 min following annealing by ramping from 95° C. to 4° C. with decreasing the temperature by 0.1° C./sec (Cobb, R. E., Wang, Y., & Zhao, H. (2015). High-Efficiency Multiplex Genome Editing of Streptomyces Species Using an Engineered CRISPR/Cas System. ACS Synthetic Biology, 4(6), 723-728).

Strains

E. coli strain Ec#098

E. coli strain Ec#098 is an E. coli INV110 strain (Life technologies) carrying the DNA-methyltransferase encoding expression plasmid pMDS003 (WO2019016051).

Generation of Bacillus licheniformis Gene k.o Strains

For gene deletion in Bacillus licheniformis strains DSM641 and ATCC 53926 (U.S. Pat. No. 5,352,604) and derivatives thereof, deletion plasmids were transformed into E. coli strain Ec#098 made competent according to the method of Chung (Chung, C. T., Niemela, S. L., and Miller, R. H. (1989). One-step preparation of competent Escherichia coli: transformation and storage of bacterial cells in the same solution. Proc. Natl. Acad. Sci. U.S.A 86, 2172-2175), following selection on LB-agar plates containing 100 μg/ml ampicillin and 30 μg/ml chloramphenicol at 37° C. Plasmid DNA was isolated from individual clones and used for subsequent transfer into Bacillus licheniformis strains. The isolated plasmid DNA carries the DNA methylation pattern of Bacillus licheniformis strains DSM641 and ATCC53926 respectively and is protected from degradation upon transfer into B. licheniformis.

B. licheniformis P304: Deleted Restriction Endonuclease

Electrocompetent Bacillus licheniformis DSM641 cells (U.S. Pat. No. 5,352,604) were prepared as described above and transformed with 1 μg of pDe1006 restrictase gene deletion plasmid isolated from E. coli Ec#098 following plating on LB-agar plates containing 5 μg/ml erythromycin at 30° C.

The gene deletion procedure was performed as described in the following:

Plasmid carrying Bacillus licheniformis cells were grown on LB-agar plates with 5 μg/ml erythromycin at 45° C. driving integration of the deletion plasmid via Campbell recombination into the chromosome with one of the homology regions of pDe1006 homologous to the sequences 5′ or 3′ of the restrictase gene. Clones were picked and cultivated in LB-media without selection pressure at 45° C. for 6 hours, following plating on LB-agar plates with 5 μg/ml erythromycin at 30° C. Individual clones were picked and screened by colony-PCR analysis with oligonucleotides SEQ ID NO. 14 and SEQ ID NO. 15 for successful genomic deletion of the restrictase gene. Putative deletion positive individual clones were picked and taken through two consecutive overnight incubation in LB media without antibiotics at 45° C. to cure the plasmid and plated on LB-agar plates for overnight incubation at 37° C. Single clones were analyzed by colony PCR for successful genomic deletion of the restrictase gene. A single erythromycin-sensitive clone with the correct deleted restrictase gene was isolated and designated Bacillus licheniformis P304.

B. licheniformis P308: deleted poly-gamma glutamate synthesis genes Electrocompetent Bacillus licheniformis P304 cells were prepared as described above and transformed with 1 μg of pDe1007 pga gene deletion plasmid isolated from E. coli INV110 cells (Life technologies) following plating on LB-agar plates containing 5 μg/ml erythromycin at 30° C.

The gene deletion procedure was performed as described for the deletion of the restrictase gene.

The deletion of the pga genes was analyzed by PCR with oligonucleotides SEQ ID NO. 17 and SEQ ID NO. 18 The resulting Bacillus licheniformis strain with deleted pga synthesis genes was named Bacillus licheniformis P308.

B. licheniformis Strain LBL: Integration of the Lux Genes Into the AmyB Locus

Electrocompetent B. licheniformis P308 were prepared as described above and transformed with 1 μg of plasmid pKS068, isolated from E. coli INV100 followed by plating on LB-agar plates containing 50 μg/ml erythromycin at 30° C.

The gene deletion procedure was performed as described for the deletion of the restrictase gene.

The deletion of the amyB gene and integration of the lux-expression cassette was analyzed by PCR with oligonucleotides SEQ ID NO. 9 and SEQ ID NO. 10 and analysis of clearing zones on LB-agar plates containing 1% starch following iodine staining with Lugol's solution. The resulting Bacillus licheniformis strain was named Bacillus licheniformis LBL.

B. licheniformis Bli#002: Deleted aprE Gene

Electrocompetent Bacillus licheniformis ATCC53926 cells were prepared as described above and transformed with 1 μg of pDe1003 aprE gene deletion plasmid isolated from E. coli Ec#098 following plating on LB-agar plates containing 5 μg/ml erythromycin at 30° C.

The gene deletion procedure was performed as described for the deletion of the restrictase gene. The deletion of the aprE gene was analyzed by PCR with oligonucleotides SEQ ID NO. 20 and SEQ ID NO. 21 The resulting Bacillus licheniformis strain with deleted aprE gene was named Bli#002.

B. licheniformis Bli#005: deleted poly-gamma glutamate synthesis genes The poly-gamma-glutamate synthesis genes were deleted in Bacillus licheniformis Bli#002 as described for the deletion of the pga genes in Bacillus licheniformis P304 with the difference that the pDe1007 plasmid was isolated from E. coli Ec#098 cells. The resulting strain was named Bli#005.

B. subtilis Strain GCX1

Bacillus subtilis 168 wild type was made competent according to the method of Spizizen (Anagnostopoulos,C. and Spizizen, J. (1961). J. Bacteriol. 81, 741-746.) and transformed with plasmid pKS099 linearized with restriction endonuclease Apal for integration of the xylR-PxylA-cop cassette together with the phleomycin resistance gene into the sacA locus. Cells were spread and incubated overnight at 37° C. on LB-agar plates containing 5 μg/ml phleomycin. Grown colonies were picked and correct integration of the expression construct into the sacA locus verified by PCR. The resulting B. subtilis strain has the genotype B. subtilis 168 sacA::xyIR-PxylA-cop-phleor and is named GCX1.

B. subtilis Strain PCX1

Bacillus subtilis 168 wild type was made competent according to the method of Spizizen (Anagnostopoulos, C. and Spizizen, J. (1961). J. Bacteriol. 81, 741-746.) and transformed with plasmid pKS111. Cells were spread and incubated overnight at 37° C. on LB-agar plates containing 20 μg/ml kanamycin. The resulting B. subtilis strain is named PCX1 and carries the replicating pKS111 plasmid.

B. subtilis Strain KDS

Bacillus subtilis 168 carrying pLS20cat Maya M. et al., 2006, Bioscience, Biotechnology, and Biochemistry, 70(3), 740-742) was made competent according to the method of Spizizen (Anagnostopoulos, C. and Spizizen, J. 1961. J. Bacteriol. 81, 741-746.) and transformed with the linearized (cut with ScaI) plasmid pBS4S (Addgene #55170 or BGSCID=ECE259) thereby replacing the thrC locus with a spectinomycin resistance cassette. Cells were spread and incubated overnight at 37° C. on LB-agar plates containing 100 μg/ml spectinomycin and 5 μg/ml chloramphenicol. Grown colonies were picked and tested for threonine auxotrophy. The resulting B. subtilis strain is threonine auxotrophic and carries the replicating pLS20cat plasmid. It was designated B. subtilis KDS.

B. subtilis Strain Bs#056

The prototrophic Bacillus subtilis strain KO-7S (BGSCID:1S145; Zeigler D. R) was made competent according to the method of Spizizen (Anagnostopoulos, C. and Spizizen, J. (1961). J. Bacteriol. 81, 741-746.) and transformed with the linearized DNA-methyltransferase expression plasmid pMIS012 for integration of the DNA-methyltransferase into the amyE gene as described for the generation of B. subtilis Bs#053 in WO2019016051. Cells were spread and incubated overnight at 37° C. on LB-agar plates containing 10 μg/ml chloramphenicol. Grown colonies were picked and stroke on both LB-agar plates containing 10 μg/ml chloramphenicol and LB-agar plates containing 10 μg/ml chloramphenicol and 0.5% soluble starch (Sigma) following incubation overnight at 37° C. The starch plates were covered with iodine containing Lugols solution and positive integration clones identified with negative amylase activity. Genomic DNA of positive clones was isolated by standard phenol/chlorform extraction methods after 30 min treatment with lysozyme (10 mg/ml) at 37° C., following analysis of correct integration of the MTase expression cassette by PCR. The resulting B. subtilis strain is named Bs#056.

B. subtilis Strain Bs#073

Bacillus subtilis 168 wild type was made competent according to the method of Spizizen (Anagnostopoulos, C. and Spizizen, J. (1961). J. Bacteriol. 81, 741-746.) and transformed with plasmid pKS102. This plasmid carries no functional origin for replication in Bacillus and can only be stably inherited when integrated into the B. subtilis 168 genome via the provided region homologous to the amyE locus. Cells were spread and incubated overnight at 37° C. on LB-agar plates containing 50 μg/ml erythromycin. Grown colonies were picked and verified by PCR. The resulting B. subtilis strain integrated the complete plasmid pKS102 into the amyE locus and is named Bs#073.

Plasmids

pEC194RS—Bacillus Temperature Sensitive Plasmid.

The plasmid pE194 is PCR-amplified with oligonucleotides SEQ ID NO. 1 and SEQ ID NO. 2 with flanking PvuII sites, digested with restriction endonuclease PvuII and ligated into vector pCE1 digested with restriction enzyme Smal. pCE1 is a pUC18 derivative, where the BsaI site within the ampicillin resistance gene has been removed by a silent mutation. The ligation mixture was transformed into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid is named pEC194S.

The type-Il-assembly mRFP cassette is PCR-amplified from plasmid pBSd141R (accession number: KY995200) (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) with oligonucleotides SEQ ID NO. 3 and SEQ ID NO.4, comprising additional nucleotides for the restriction site BamHI. The PCR fragment and pEC194S were restricted with restriction enzyme BamHI following ligation and transformation into E. coli DH10B cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting plasmid pEC194RS carries the mRFP cassette with the open reading frame opposite to the reading frame of the erythromycin resistance gene.

pEC194RS(ts)—Bacillus Stringent Temperature Sensitive Plasmid

Two single nucleotide exchanges were introduced into the pE194 origin of replication of pEC194RS using the QuikChange™ Kit (Agilent) and oligonucleotides SEQ ID NO. 64 and SEQ ID NO. 65. These mutations correspond to the temperature sensitivity mutations described by Villafane et al. (Villafane, R. et al. 1987. Replication control genes of plasmid pE194. Journal of bacteriology, 169(10), 4822-4829.). Plasmid DNA was isolated from individual clones and analyzed for correctness by sequencing. The resulting sequence verified plasmid was named pEC194RS(ts).

pEC194RSdelta(cop-repF)—Non-Replicative pEC194RS Derivative

pEC194RSdelta(cop-repF) was used as an integrative control and is non-replicative in Bacillus due to the deletion of the cop-repF operon. The backbone of pEC194RS was amplified with oligonucleotides SEQ ID NO. 28 and SEQ ID NO. 29, flanked by NcoI restriction sites. The backbone fragment was cut with NcoI and re circularized by self-ligation. The reaction mixture was subsequently transformed into E. coli DH10ß cells (Life technologies). Transformants were spread and incubated overnight at 37° C. on LB-agar plates containing 100 μg/ml amplicillin. Plasmid DNA was isolated from individual clones and analyzed for correctness by sequencing. The resulting sequence verified plasmid was named pEC194RSdelta(cop-repF).

pDe1003—aprE Gene Deletion Plasmid

The gene deletion plasmid for the aprE gene of Bacillus licheniformis was constructed with plasmid pEC194RS and the gene synthesis construct SEQ ID NO. 19 comprising the genomic regions 5′ and 3′ of the aprE gene flanked by BsaI sites compatible to pEC194RS. The type-II-assembly with restriction endonuclease BsaI was performed as described (Radeck, J., Meyer, D., Lautenschlager, N., and Mascher, T. 2017. Bacillus SEVA siblings: A Golden Gate-based toolbox to create personalized integrative vectors for Bacillus subtilis. Sci. Rep. 7: 14134) and the reaction mixture subsequently transformed into E. coli DH10ß cells (Life technologies). Transformants were spread and incubated overnight at 37C on LB-agar plates containing 100 μg/ml ampicillin. Plasmid DNA was isolated from individual clones and analyzed for correctness by restriction digest. The resulting aprE deletion plasmid is named pDe1003.

pDe1006—Restrictase Gene Deletion Plasmid

The gene deletion plasmid for the restrictase gene (SEQ ID NO. 12) of the restriction modification system of Bacillus licheniformis DSM641 (SEQ ID NO. 11) was constructed with plasmid pEC194RS and the gene synthesis construct SEQ ID NO. 13 comprising the genomic regions 5′ and 3′ of the restrictase gene flanked by BsaI sites compatible to pEC194RS. The type-II-assembly with restriction endonuclease BsaI was performed as described above and the reaction mixture subsequently transformed into E. coli DH10ß cells (Life technologies). Transformants were spread and incubated overnight at 37° C. on LB-agar plates containing 100 μg/ml ampicillin. Plasmid DNA was isolated from individual clones and analyzed for correctness by restriction digest. The resulting restrictase deletion plasmid is named pDe1006.

pDe1007—Poly-Gamma-Glutamate Synthesis Genes Deletion Plasmid

The deletion plasmid for deletion of the genes involved in poly-gamma-glutamate (pga) production, namely ywsC (pgsB), ywtA (pgsC), ywtB (pgsA), ywtC (pgsE) of Bacillus licheniformis was constructed as described for pDe1006, however the gene synthesis construct SEQ ID NO. 16 comprising the genomic regions 5′ and 3′ flanking the ywsC, ywtA (pgsC), ywtB (pgsA), ywtC (pgsE) genes flanked by BsaI sites compatible to pEC194RS was used. The resulting pga deletion plasmid is named pDe1007.

Plasmid p#0074—xylR-PxylA Promoter Fragment

The Bacillus megaterium DSM319 promoter PxylA of the xylA gene and the xylose repressor gene xylR located 5′ to the PxylA in opposite direction was ordered as gene synthesis construct optimized for low content of restriction endonuclease sites and flanked by type-II restriction endonuclease BpiI sites (SEQ ID NO. 22).

Plasmid pBW424—Low Copy Bacillus T2A—E. coli Shuttle Vector

The plasmid pBW424 is a low-copy Bacillus-E. coli shuttle vector based on the pBS72 origin of replication (Titok, M. A. et al. 2003, Plasmid, 49(1), 53-62.) with the kanamycin resitance gene of pUB110, a ColE1 origin of replication for E. coli and a type-II-assembly cassette for subsequent cloning of expression construct. The plasmid was constructed as described below with gene synthesis constructs optimized for low numbers of restriction endonuclease sites and flanked by restriction endonuclease BsaI sites.

SEQ ID NO. 68 comprises the origin of replication of pBS72 (accession number: AY102630) which has been optimized for less endonuclase restriction sites compared to the published sequence. SEQ ID NO. 69 comprises the modified mRFP cassette from plasmid pBSd141R (accession number: KY995200; Radeck et al., 2017; Sci. Rep. 7: 14134) with flanking type-II restriction enzyme sites of BpiI, the terminator region of the aprE gene from Bacillus licheniformis.

SEQ ID NO. 70 comprises a modified kanamycin resistances gene of pUB110 and a modified ColE1 origin of replication. The plasmid pBW424 was assembled by type-II restriction cloning described by Radeck et al. 2017 using BsaI. The ligation mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by restriction digest following the sequencing. The resulting plasmid is named pBW424.

Plasmid p#692—Low Copy Bacillus T2A—E. coli Shuttle Vector with Strong Terminator

The t1t2t0 terminator (derived from pMUTIN) was introduced 5′ the type-II-assembly cassette of pBW424 to prevent potential read-through from the kanamycin selection marker.

The terminator sequence t1t2t0 was integrated into pBW424 by Gibson assembly (NEBuilder® HiFi DNA Assembly Cloning Kit, New England Biolabs). To this purpose, the terminator fragment (0.44 kb) was amplified by PCR with oligonucleotides SEQ ID NO. 71 and SEQ ID NO. 72 using pMutin2 (accession number AF072806) as the template. The corresponding vector backbone of pBW424 was amplified with oligonucleotides SEQ ID NO. 73 and SEQ ID NO. 74. The pBW424 amplicon was purified using the PCR product purification kit (Roche). After subsequent digestion of the pBW424 PCR product with DpnI (New England Biolabs), both PCR fragments were gel purified using the Qiaquick Gel Extraction Kit (Qiagen, Hilden, Germany) and annealed in a 1:2 ratio for 1 h at 50° C. E. coli strain DH10B was transformed with the assembly reaction following plating on LB-agar plates containing 20 μg/ml kanamycin. Plasmid DNA was isolated from individual clones and analyzed for correctness by restriction digest and sequencing. The resulting plasmid is named p#692.

Plasmid p#0268—Cargo Vector for Type-II-Assembly

Plasmid p#0268 comprises a gene synthesis construct (SEQ ID NO. 23) and is a derivative of the cargo vector derivatives of pSEVA243 (Radeck et al., 2017; Sci. Rep. 7(1): 14134) comprising the lacZ gene flanked by restriction endonuclease BpiI sites and terminator regions 5′ and 3′ to protect cloned expression cassettes from potential transcriptional acitivties. This modified lacZ cassette is again flanked by type-II restriction sites BsaI, BsmBI, AarI RE sites according to type-II-assembly concept of Radeck et al. 2017.

Plasmid p#0268ter—Cargo Vector for Type-II-Assembly Including an Upstream Terminator

Plasmid p#0268ter was used as a cargo vector and includes a terminator upstream of the insert. Plasmid p#0268 was cut with BpiI, a terminator-mRFP fragment PCR-amplified from p#0692 with oligonucleotides SEQ ID NO. 77 and SEQ ID NO. 78 and subsequently cleaved with restriction endonuclease BsaI. The two parts were ligated and the ligation mixture was transformed into E. coli DH10B cells (Life technologies). Transformants were spread and incubated overnight at 37° C. on LB-agar plates containing 20 μg/ml kanamycin. Plasmid DNA was isolated from individual red clones and analyzed for correctness by sequencing. The resulting plasmid is named p#268ter.

Plasmid p#0268cat—Cargo Vector with 3′ Chloramphenicol Resistance Gene

Plasmid p#0268cat was used as a cargo vector and includes a chloramphenicol resistance cassette downstream of the insert. p#0268 was cleaved with restriction endonuclease SmaI at the unique SmaI site and ligated with a PCR fragment, containing a chloramphenicol resistance cassette that was PCR-amplified from pBS3Clux (Radeck et al., J Biol Eng. 2013 Dec. 2; 7(1):29) using oligonucleotides SEQ ID NO. 51 and SEQ ID NO. 52. The ligation mixture was transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by sequencing. The resulting plasmid is named p#268cat.

Plasmid p#0268ter-cat—Cargo Vector with 5′ Terminator with 3′ Chloramphenicol Resistance Gene

Plasmid p#0268ter-cat was used as a cargo vector and includes a terminator upstream and a chloramphenicol resistance cassette downstream of the insert. Plasmid p#0268cat was cut with BpiI and a terminator-mRFP fragment was PCR-amplified from p#692 with oligonucleotides SEQ ID NO. 77 and SEQ ID NO. 78 and subsequently cleaved with restriction endonuclease BsaI. The two parts were ligated and the ligation mixture was transformed into E. coli DH10B cells (Life technologies). Transformants were spread and incubated overnight at 37° C. on LB-agar plates containing 20 μg/ml kanamycin. Plasmid DNA was isolated from individual red clones and analyzed for correctness by sequencing. The resulting plasmid is named p#268ter-cat.

Plasmid p#0268phleo—Cargo Vector with 3′ Phleomycin Resistance Gene

Plasmid p#0268phleo was used as a cargo vector and includes a phleomycin resistance cassette downstream of the insert. p#0268 was cleaved at the unique SmaI site and ligated with the phleomycin resistance cassette that was PCR-amplified from pBSc243B (Radeck et al., 2017; Sci. Rep. 7(1): 14134) with oligonucleotides SEQ ID NO. 49 and SEQ ID NO. 50. The ligation mixture was transformed into E. coli DH10? cells (Life technologies). Transformants were spread and incubated overnight at 37° C. on LB-agar plates containing 20 μg/ml kanamycin. Plasmid DNA was isolated from individual clones and analyzed for correctness by restriction digest following the sequencing. The resulting plasmid is named p#268phleo.

Plasmid p#0268ter-phleo—Cargo Vector with 5′ Terminator and with 3′ Phleomycin Resistance Gene

Plasmid p#0268ter-phleo was used as a cargo vector and includes a terminator upstream and a phleomycin resistance cassette downstream of the insert. Plasmid p#0268phleo was cleaved with restriction endonuclease BpiI and a terminator-mRFP fragment PCR-amplified from plasmid p#0692 with oligonucleotides SEQ ID NO. 77 and SEQ ID NO. 78) was cleaved with restriction endonuclease BsaI. The two parts were ligated together and the ligation mixture was transformed into E. coli DH10? cells (Life technologies). Transformants were spread and incubated overnight at 37° C. on LB-agar plates containing 20 μg/ml kanamycin. Plasmid DNA was isolated from individual red clones and analyzed for correctness by sequencing. The resulting plasmid is named p#268ter-phleo.

Plasmid pKS099—Plasmid for Integtration of xylR-PxylA-cop Expression Cassette Into sacA Locus of Bacillus subtilis

The genomic integration plasmid pKS099 was used for the insertion of xylR-PxylAcop into the sacA gene of Bacillus subtilis and was constructed in a two-step process. First, the xylR-PxylA fragment of plasmid p#0074 (SEQ ID NO. 22) was assembled with the cop coding region of pE194RS PCR-amplified using oligonucleotides SEQ ID NO. 59 and SEQ ID NO. 60 into the cargo vector p#0268ter-phleo by a type-II-assembly reaction with BpiI restriction endonuclease. The reaction mixture was transformed into E. coli DH10? cells (Life technologies). Transformants were spread and incubated overnight at 37° C. on LB-agar plates containing 20 μg/ml kanamycin. Plasmid DNA was isolated from individual clones and analyzed for correctness by sequencing. The resulting plasmid was assembled with genomic regions 5′ and 3′ of the sacA gene (amplified from the B. subtilis genome using oligonucleotides SEQ ID NO. 53 and SEQ ID NO. 54 and SEQ ID NO. 55 and SEQ ID NO. 56) and pBSd191R (Radeck et al., 2017; Sci. Rep. 7: 14134; BGSC ID: ECE702) backbone vector, all flanked by BsaI sites. The second type-II-assembly with restriction endonuclease BsaI was performed as described and the reaction mixture subsequently transformed into E. coli DH10? cells. Transformants were spread and incubated overnight at 37° C. on LB-agar plates containing 100 μg/ml ampicillin. Plasmid DNA was isolated from individual clones and analyzed for correctness by sequencing. The resulting plasmid was named pKS099.

Plasmid pKS111: High-Copy E. coli—Low-Copy Bacillus Vector for Cop Expression

Plasmid pKS111 serves as a low-copy expression vector in Bacillus. for the expression of the cop gene of pE194 under the control of the xylose inducible promoter PxylA from Bacillus megaterium. pKS111 was constructed with T2A-assembly with restriction endonuclease BpiI with the following components: plasmid p#0692 as destination vector, the xylR-PxylA fragment of plasmid p#0074 (SEQ ID NO. 22) and the cop fragment amplified from pEC194RS using oligonucleotieds SEQ ID NO. 59 and SEQ ID NO. 60. The reaction mixture was transformed into E. coli DH10? cells. Transformants were spread and incubated overnight at 37° C. on LB-agar plates containing 20 μg/ml kanamycin. Plasmid DNA was isolated from individual clones and analyzed for correctness by sequencing. The resulting plasmid was named pKS111.

Plasmid pBS3KcatluxGG

The plasmid pBS3KcatluxGG served as a template for the PCR amplification of the type II assembly compatible luxABCDE operon. All BsaI, BsmBI and AarI restriction enzyme recognition sites inside the luxABCDE operon of the vector pBS3Kcatlux (Popp, P. F. et al. 2017. 7(1), 1-13.) were removed by introducing silent mutations using the QuikChange™ Kit (Agilent) and oligonucleotides SEQ ID NO. 46, SEQ ID NO. 47 and SEQ ID NO. 48. The resulting vector with functional luciferase activity was designated pBS3KcatluxGG.

Plasmid p#0268ter-Pveglux—Pveg-luxABCDE

Plasmid p#0268ter-Pveglux combines the luciferase reporter genes with the constitutive Pveg promoter from Bacillus subtilis flanked by T2A restriction endonuclease sites. The Pveg fragment was PCR-amplified from TMB3090 (Popp, P. F. et al. 2017. The Bacillus BioBrick Box 2.0: expanding the genetic toolbox for the standardized work with Bacillus subtilis. Scientific reports, 7(1), 1-13.). using oligonucleotides SEQ ID NO 062 and SEQ ID NO 063 with BpiI site overhangs. The reporter genes luxABCDE were PCR-amplified with oligonucleotides SEQ ID NO. 57 and SEQ ID NO. 58 from pBS3KcatluxGG. Plasmid p#0268ter served as a cargo vector. This intermediate promoter-reporter cargo plasmid was constructed by a type-II-assembly reaction with restriction endonuclease BpiI as described (Radeck et al., 2017; Sci. Rep. 7(1): 14134) and the reaction mixture was subsequently transformed into E. coli DH10B cells (Life technologies). Transformants were spread and incubated overnight at 37° C. on LB-agar plates containing 20 μg/ml kanamycin. Plasmid DNA was isolated from individual clones and analyzed for correctness by sequencing. The resulting sequence verified plasmid was named p#0268ter-Pveglux.

Plasmid pKS100—Luminescence Plasmid Based on pEC194RS for Use in B. subtilis

Plasmid pKS100 is based on plasmid pEC194RS carrying in addition a 600 bp fragment homologous to the B. subtilis amylase amyE gene and the luciferase gene under the control of the Pveg promoter from plasmid p#0268ter-Pveglux. The amylase gene fragment was PCR-amplified with oligonucleotides SEQ ID NO 066 and SEQ ID NO 067. The 5′ phosphorylated oligonucleotides SEQ ID NO. 79 and 080 were annealed to form an oligonucleotide duplex. Plasmid pKS100 was constructed by type-Il-assembly with BsaI restriction endonuclease as described above with the following compontents: pEC194RS, PCR-fragment amyE, oligonucleotide duplex and p#0268ter-Pveglux. The reaction mixture was transformed into E. coli DH10B cells (Life technologies) and subsequently transformants were spread and incubated overnight at 37° C. on LB-agar plates containing 100 μg/ml ampicillin. Plasmid DNA was isolated from individual clones and analyzed for correctness by restriction digest and sequencing. The resulting plasmid was named pKS100.

Plasmid pKS101—Luminescence Plasmid Based on pEC194RS(ts) for Use in B. subtilis

pKS101 was constructed as described for plasmid pKS100, however plasmid pEC194RS(ts) was used.

Plasmid pKS102—Luminescence Integration Plasmid in B. subtilis

pKS102 was constructed as described for plasmid pKS100, however plasmid pEC194RS?(cop-repF) was used.

Plasmid pKS200—Luminescence Plasmid Based on pEC194RS for Use in B. lichenformis Plasmid pKS200 was constructed as described for plasmid pKS100, however instead of B. subtilis amyE fragment, a fragment homologous to 600 bp of the B. licheniformis amyB gene was PCR-amplified from the B. licheniformis P308 genome using oligonucleotides SEQ ID NO. 75 and SEQ ID NO. 76.

Plasmid p#0268-PsrfAAlux—PsrfAAluxABCDE Cargo

Plasmid p#0268-PsrfAAlux was constructed as described for plasmid p#0268ter-Pveglux with the following differences. Instead of the Pveg promtoter, the promoter of the srfAA gene from Bacillus subtilis was PCR-amplified from B. subtilis genomic DNA with oligonucleotides SEQ ID NO. 30 and SEQ ID NO. 31, carrying BpiI restriction sites. The reporter gene operon luxABCDE was PCR-amplified with oligonucleotides SEQ ID NO. 57 and SEQ ID NO. 61 from pBS3KcatluxGG.

Plasmid pKS068—luxABCDE Integration amyB Plasmid

Plasmid pKS068 was used for integration of the luxABCDE expression cassette into the amylase amyB gene locus of B. licheniformis. The 5′ and 3′ regions of the amyB gene of B. licheniformis were PCR-amplified from B. licheniformis genomic DNA with oligonucleotides SEQ ID NO. 5, SEQ ID NO. 6 and SEQ ID NO. 7, SEQ ID NO. 8, respectively, comprising flanking Bsal endonuclease restriction sites. Plasmid pKS068 was constructed by type-II-assembly as described above with plasmid pEC194RS, 5′ amyB homology region, plasmid PsrfAAluxABCDE and 3′ amyB homology region. The reaction mixture was transformed into E. coli DH10R cells (Life technologies) and subsequently transformants were spread and incubated overnight at 37° C. on LB-agar plates containing 100 μg/ml ampicillin. Plasmid DNA was isolated from individual clones and analyzed for correctness by restriction digest and sequencing. The resulting plasmid was named pKS068.

Plasmid pBAio—Bacillus All-in-One Vector

The pBAio plasmid is an E. coli—Bacillus shuttle vector with the temperature-sensitive replication orgin of plasmid pEC194RS, a kanamycin resistance cassette functional in E. coli and Bacillus and an additional copy of the cop gene under control of the Bacillus megaterium PxylA promoter. Plasmid pBAio is composed of the following genetic elements:
i) Kanamycin resistance gene and ColE1 replication origin were PCR-amplified from p#0692 using oligonucleotides SEQ ID NO. 34 (Ncol) and SEQ ID NO. 35 (PstI),
ii) a terminator fragment was amplified from pKS111 using SEQ ID NO. 38 (SpeI) and SEQ ID NO. 39 (BamHI),
iii) a xylR-PxylA-cop fragment was PCR-amplified from pKS111 using SEQ ID NO. 44 (NsiI) and SEQ ID NO. 45 (SpeI),
iv) the type-II-assembly cassette was PCR-amplified from pEC194RS using SEQ ID NO. 36 (BamHI) and SEQ ID NO. 37 (NcoI),
v) an oriT fragment was PCR-amplified from pLS20 (ltaya M. et al., 2006, Bioscience, Biotechnology, and Biochemistry, 70(3), 740-742) using SEQ ID NO. 42 (NcoI) and SEQ ID NO. 43 (XbaI) and
vi) a pE194 origin of replication PCR-amplified from from pEC194RS using SEQ ID NO. 40 (SpeI) and SEQ ID NO. 41 (NcoI).

Fragments KanR+ColE1 (i), xylR PxylA-cop (ii), terminator (iii) and type-Il-assembly cassette (iv) were cleaved at the designated restriction sites and ligated to form an intermediate construct (pBAioint). The reaction mixture was transformed into E. coli DH10R cells and subsequently transformants were spread and incubated overnight at 37° C. on LB-agar plates containing 20 μg/ml kanamycin. Plasmid DNA was isolated from individual clones and analyzed for correctness by restriction digest and sequencing. This intermediate plasmid (pBAioint) was cleaved at the unique NcoI site, treated with phosphatase (rSAP by NEB) and purified via an SLG DNA clean-up Kit (SLG), following ligation with with the fragments oriT (v) and pE194 origin of replication (vi). The reaction mixture was transformed into E. coli DH10R cells and subsequently transformants were spread and incubated overnight at 37° C. on LB-agar plates containing 20 μg/ml kanamycin. Plasmid DNA was isolated from individual clones and analyzed for correctness by restriction digest and sequencing. The resulting plasmid was designate pBAio.

pBAio-lumiBs—Luminescence Plasmid Based on pBAio for Use in B. subtilis

Plasmid pBAio-lumBs was constructed as described for pKS100, however instead of the plasmid pEC194RS the plasmid pBAio was used.

pBAio-lumiBI—Luminescence Plasmid Based on pBAio for Use in B. licheniformis

Plasmid pBAio-lumBI was constructed as described for pKS200, however instead of the plasmid pEC194RS the plasmid pBAio was used.

Plasmid pKS137—luxA Integration Plasmid Based on pBAio

Plasmid pKS137 was used for Campbell-based recombination/integration into the luxA gene of the luxABCDE operon for functional inactivation. Plasmid pKS137 was constructed with plasmid pBAio and a 500 bp region homologous to luxA (PCR-amplified from pBS3KcatluxGG using SEQ ID NO. 32 and SEQ ID NO. 33) flanked by BsaI sites compatible to pBAio. The type-II-assembly with the restriction endonuclease BsaI was performed as described above and the reaction mixture was subsequently transformed into E. coli DH10B cells. Transformants were spread and incubated overnight at 37° C. on LB-agar plates containing 20 μg/ml kanamycin. Plasmid DNA was isolated from individual clones and analyzed for correctness by sequening. The resulting luxA gene integration plasmid is named pKS137.

### EXAMPLES

### Example 1

### Temperature-Dependent Plasmid Copy Number of pEC194RS and pEC194RS(ts) Derivatives

The temperature-dependent copy numbers of plasmids that derive from pE194 and pE194(ts) are determined by real-time quantitative PCR.

Bacillus subtilis 168 strains carrying plasmids pKS100 (based on pEC194RS), pKS101 (based on pEC194RS(ts)) and the Bacillus subtilis reference strain Bs#073 which carries the pKS102 sequence (lacking the cop-repF operon) integrated into the genome were cultivated in 2 ml LB-Lennox media with 50 μg/ml erythromycin for 16 h at 30° C. with agitation. The next day, the cultures were diluted to an OD600 of 0.1 in LB-Lennox media supplemented with erythromycin (50 μg/ml) and divided into three shake flasks with 10 ml each, following incubation at 30° C., 37° C. and 42° C. respectively. After 8 h of cultivation, when cells reached transition phase, samples were withdrawn and cells were recovered by centrifugation.

Total DNA was isolated from the individual samples and the plasmid copy number determined by real-time quantitative PCR (qTower³, AnalytikJena using the Luna® Universal qPCR Master Mix, NEB). Oligonucleotides SEQ ID NO. 26 and SEQ ID NO. 27 within the bla gene encoding for the ampicillin resistance gene on the pEC194RS and pEC194RS(ts) derivatives and oligonucleotides SEQ ID NO. 24 and SEQ ID NO. 25 within the rtp locus of Bacillus subtilis (locus tag BSU_18490) in the vicinity of the chromosomal terminus were used for RT-qPCR amplification. Purified plasmid DNA and purified chromosomal DNA served as measure for absolute quantitation for the calculation of the PCR efficiencies, which were 99% and 98%, respectively.

The average amount of plasmids per cell was calculated relative to the amounts of chromosomal termini, the latter representing a single cell with one chromosome.

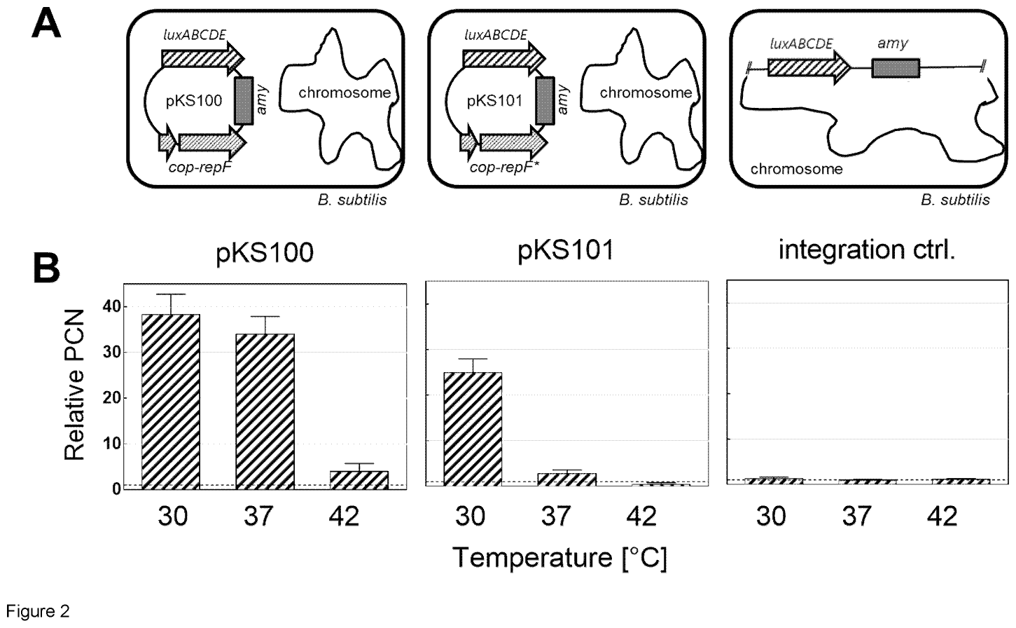

The result is depicted in FIG. 2. Bacillus subtilis strain Bs#073 with integrated non-replicative plasmid backbone serves as single copy control under all temperature conditions.

Plasmid pKS100 has an average copy number of 38.5 and and 34.1 at 30° C. and 37° C. respectively and a strongly reduced average copy nuber of 4.1 at the non-permissive temperature 42° C. Plasmid pKS101 with the more stringent temperature sensitive origin of replication has an average copy number of 25 plasmids at 30° C. and an average number of 2.9 copies per cell at 37° C. At 42° C. the average plasmid copy number is calculated below 1 which indicates plasmid loss. Cells without plasmid are not viable under antibiotic selective pressure, however contribute their genomic DNA as detected by qPCR analysis.

### Example 2

### Decrease of Plasmid Copy Number—Expression of Additional Chromosomal Copy of the Cop Gene

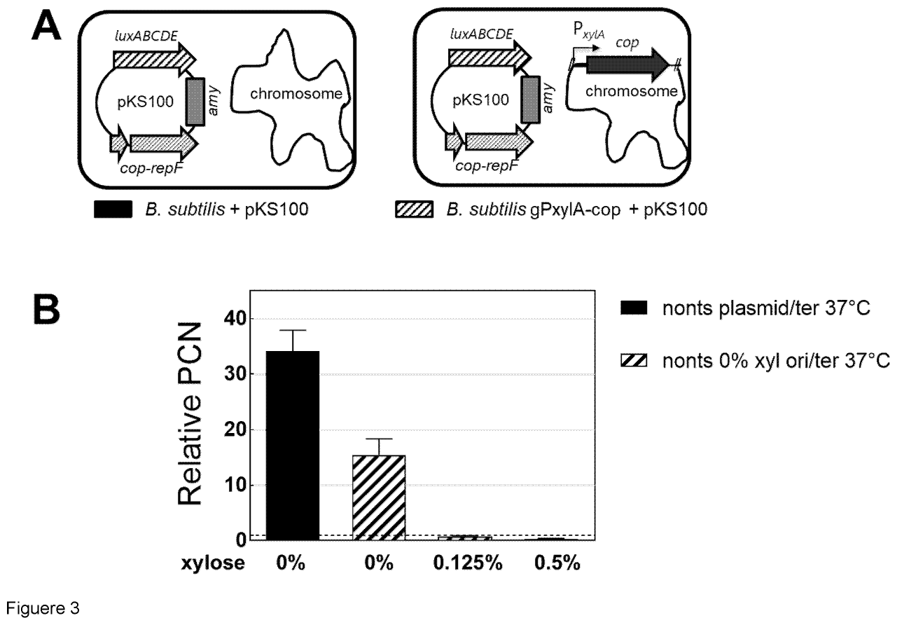

B. subtilis strain GCX1 carries the cop gene expression cassette integrated in the sacA locus. The transcription of the chromosomally located cop gene is under control of the xylose inducible promoter PxylA from Bacillus megaterium, hence cop expression is controlled in a xylose-dependent manner. B. subtilis GCX1 was transformed with plasmid pKS100 and a single clone recovered after overnight incubation on LB agar plates containing erythromycin at 37° C. Single clones of B. subtilis strain 168 carrying pKS100 (see example 1) and B. subtilis strain GCX1 carrying plasmid pKS100 were used to inoculate 2 ml LB-Lennox media with 50 μg/ml erythromycin following cultivation for 16 h at 37° C. with agitation. The next day, the cultures were diluted to an OD600 of 0.1 in LB-Lennox media supplemented with 50 μg/ml erythromycin. 10 ml of the culture of B. subtilis strain 168 carrying pKS100 was transferred into a new 100 ml shake flask and incubated with agitation at 37° C. The culture of B. subtilis strain GCX1 with plasmid pKS100 was divided into three 100 ml shake flasks with 10 ml each, supplemented with 0%, 0.125% or 0.5% xylose and incubated with agitation at 37° C. Samples were withdrawn after 8 h and cells were recovered by centrifugation. Total DNA was isolated from the individual samples and the average plasmid copy number per cell determined as described under example 1. The result is depicted in FIG. 3.

In B. subtilis 168 strain plasmid pKS100 has an average plasmid copy number of 34. In contrast, plasmid pKS100 has a lower average copy number of 15.4 copies per cell in B. subtilis strain GCX1 with the additional copy of the cop gene located in the genome even in the absence of inducer molelule xylose. The lower average copy number of pKS100 in B. subtilis strain GCX1 indicates low-level, leaky expression of the cop gene from the PxylA promoter in the absence of inducer molecule xylose. In the presence of 0.125% or 0.5% xylose in the culture medium, the elevated induced cop expression levels sufficiently block plasmid replication as seen by the average copy number of 1 or below 1 respectively.

### Example 3

### Decrease of Plasmid Copy Number—Expression of Additional Chromosomal Copy of the Cop Gene

Plasmids pKS100 and pKS101 carry a 600 bp region homologous to the amylase amyE gene of B. subtilis and carry the luxABCDE genes as reporter system which allows quantification by luminescence measurement. As the expression of the luxABCDE genes is under the control of the constitutive Pveg promoter of B. subtilis, a decrease/increase of luminescence signal directly reflects changes in plasmid copy number.

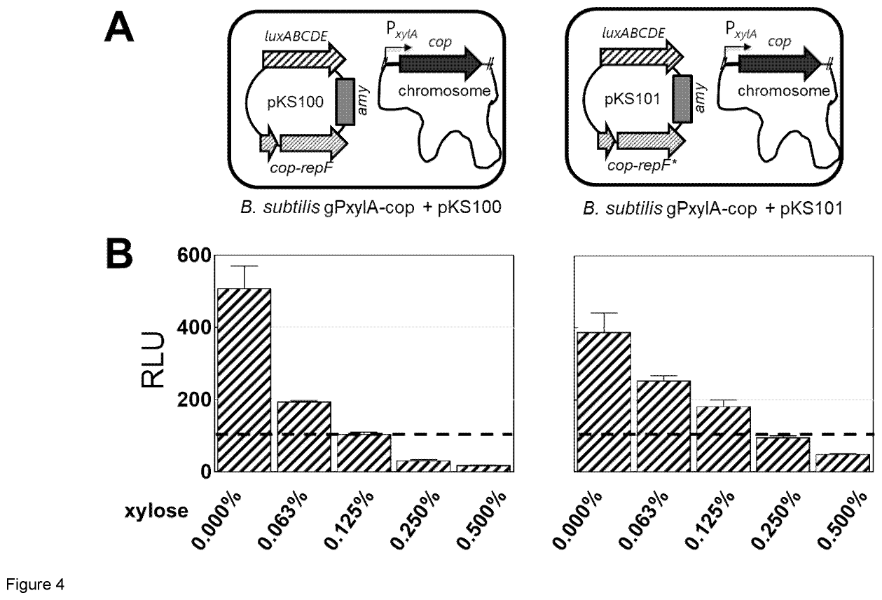

B. subtilis strains GCX1 carrying plasmid pKS100 (pE194 origin of replication) and plasmid pKS101 (pE194(ts) origin of replication) and reference B. subtilis strain Bs#073 with single-copy integrated luxABCDE genes (via pKS102) were used to inoculate 2 ml LB-Lennox medium with 50 μg/ml erythromycin following cultivation for 16 h at 30° C. with agitation. The next day, the cultures were diluted to an OD600 of 0.1 in LB-Lennox medium supplemented with erythromycin (50 μg/ml). 200 μl culture each was transferred to a black 96 well microtiter plate (Sarstedt) and supplemented with xylose to final concentrations of 0%, 0.0625%, 0.125%, 0.25% or 0.5% xylose. The cells were incubated in a Synergy Neo2 HTS Multi-Mode Microplate Reader (Bio-Tek) at 30° C. with medium agitation. The OD600 and luminescence was measured after 8 h of cultivation. The relative luminescence units (RLU) as luminescence signal per OD600 at the 8 h timepoint is determined and plotted against the indicated xylose concentrations (FIG. 4). The RLU of the single-copy control strain Bs#073 with integrated lux genes is indicated as dotted line.

With the addition of the inducer molecule xylose, cop gene expression is activated from the genomically integrated cop gene, the resulting lower levels of the RLU signal for both plasmids pKS100 and pKS101 reflect lower plasmid copy numbers. Concentrations of 0.25% and 0.50% xylose inducer molecule lower RLU signals below the Bs#073 integration control (dotted line in FIG. 4) which reflects plasmid loss within the cell population. Lower cop expression of the genomically integrated cop gene (0.063%, 0.125% xylose inducer molecule) show stable lower plasmid copy numbers for plasmids pKS100 and pKS101 in reference to absence of inducer molecule xylose.

This shows that the reduction of plasmid copy number is tuneable dependent on the xylose inducer molecule concentration.

### Example 4

### Decrease of Plasmid Copy Number—Expression of the Cop Gene from Second Plasmid

Similar to example 3, the additional cop gene was placed unter the control of the inducible PxylA promoter, however, the additional cop expression cassette is encoded on the co-resident low-copy plasmid pKS111.

B. subtilis strain PCX1 (carrying pKS111) was transformed with plasmids pKS100 and pKS101 respectively and incubated on LB-Lennox agar plates containing 50 μg/ml erythromycin and 20 μg/ml kanamycin at 30° C. As control B. subtills 168 strain with plasmid pKS100 was used. Single clones were picked and cultivated in 2 ml LB-Lennox media for 16 h at 30° C. with agitation. For B. subtilis 168 strain background 50 μg/ml erythromycin and for B. subtilis PCX1 strain background 50 μg/ml erythromycin and 20 μg/ml kanamycin were added to the media. The next day, the cultures were diluted to an OD600 of 0.1 in LB-Lennox media supplemented with corresponding antibiotics. 200 μl culture each was transferred to a black 96 well microtiter plate (Sarstedt) and supplemented with xylose to final concentrations of 0%, 0.0625%, 0.125%, 0.25% or 0.5% xylose. The cells were incubated in a Synergy Neo2 HTS Multi-Mode Microplate Reader (BioTek) at 30° C. with medium agitation. The OD600 and luminescence were measured after 8 h of cultivation.

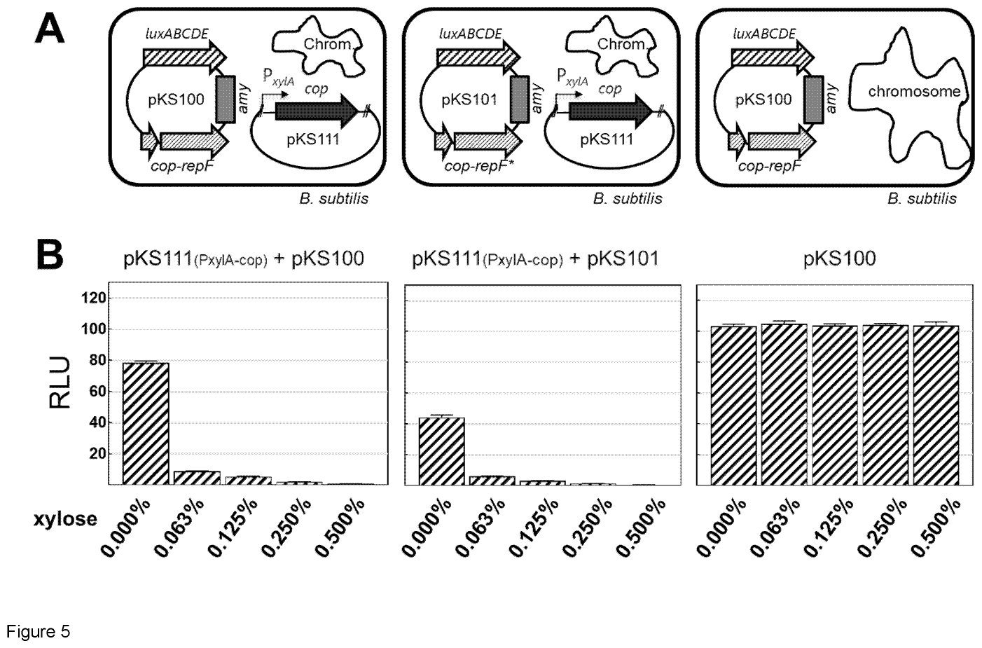

The relative luminescence units (RLU) as luminescence signal per OD600 at the 8h timepoint is determined and plotted against the indicated xylose concentrations (FIG. 5).

Reference strain B. subtilis 168 with plasmid pKS100 shows comparable RLU signals irrespective the absence or addition of increasing concentrations of inducer molecule xylose. The RLU signal of B. subtilis strain PCX1 with plasmid pKS100 without inducer molecule is lower compared to the signal from the reference strain. This indicates leaky expression of the cop gene which already results lower RLU signals (approximately 20%) indicative of lower plasmid copy number. Increasing cop expression by addition of increasing amounts of inducer molecule xylose lowers the RLU signals in a xylose concentration dependent manner. The same dependency is observed for B. subtilis PCX1 strain with plasmid pKS101, albeit at lower starting level. This is in agreement with lower plasmid copy number of pKS101 in comparison to pKS100 (Example 1, FIG. 2).

In comparison to example 3, where B. subtilis strain GCX1 with genomically integrated cop expression cassette was used, the effect of the plasmid-based additional copies of the cop expression cassette can be observed. Plasmid pKS111 with the pBS72 replication origin has approximately 6 copies per cell (Titok, M. A. et al, 2003. Plasmid, 49(1), 53-62.). This plasmid copy effect results in approximately 6-fold higher cop expression at the given xylose concentrations and therefore the impact on lower RLU signals, hence lower plasmid copy numbers of plasmid pKS100 and pKS101 can be observed.

### Example 5

### Decrease of Plasmid Copy Number with Expression of an Additional Cop Gene From the Same Plasmid—pBAio in B. subtilis

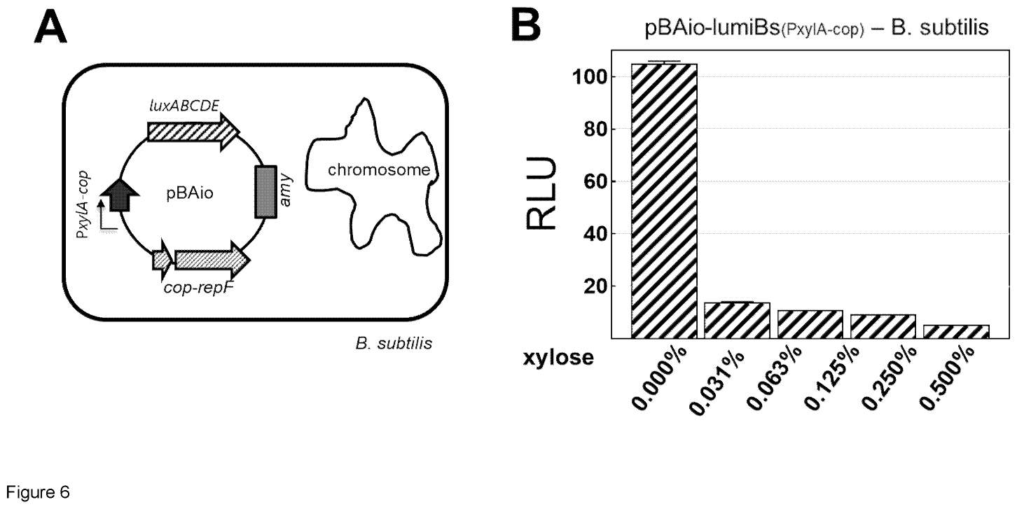

Plasmid pBAio-lumiBs is functionally similar to pKS100, but carries in addition an additional cop gene under the control of the PxylA promoter as described in examples 2, 3 and 4. B. subtilis 168 strain was transformed with pBAio-lumiBs and was cultivated on LB agar plates containing 20 μg/ml kanamycin at 30° C. A single clone was picked and cultivated in 2 ml LB-Lennox medium with 20 μg/ml kanamycin for 16 h at 30° C. with agitation. The next day, the culture was diluted to an OD600 of 0.1 in LB-Lennox media supplemented with 20 μg/ml kanamycin. 200 μl culture each was transferred to a black 96 well microtiter plate (Sarstedt) and supplemented with xylose to final concentrations of 0%, 0.0313%, 0.0625%, 0.125%, 0.25% or 0.5% xylose. The cells were incubated in a Synergy Neo2 HTS Multi-Mode Microplate Reader (BioTek) at 30° C. with medium agitation. The OD600 and luminescence was measured after 8 h of cultivation. The relative luminescence units (RLU) as luminescence signal per OD600 at the 8 h timepoint is determined and plotted against the indicated xylose concentrations (FIG. 6). The change of plasmid copy number is reflected from the change of the luminescence output of Pveg-lux, encoded on plasmid pAio-lumiBs. Increased expression of the additional cop gene on plasmid pBAio-lumiBs with increasing concentrations of inducer molecule xylose lowers the RLU signal, hence lowers the plasmid copy number of plasmid pBAio-lumiBs. Addition of 0.0313% xylose results in a 7-fold lower RLU signal (15% of non-induced control) of plasmid pBAio-lumiBs.

### Example 6

### Decrease of Plasmid Copy Number with Expression of an Additional Cop Gene from the Same Plasmid—pBAio in B. licheniformis

Plasmid pBAio-lumiBI is functionally identical to pBAio-lumiBs with the additional cop gene present on pBAio-lumiBI, but carrying a 600 bp fragment homologous to the amylase amyB locus of B. licheniformis.

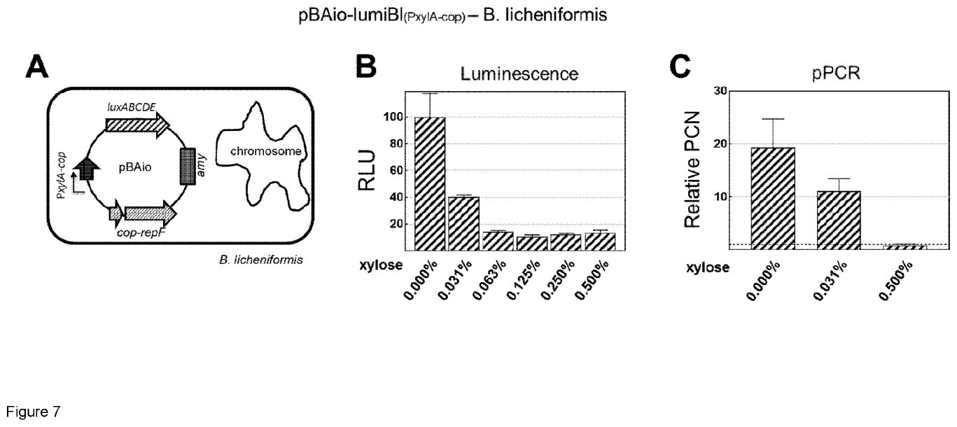

B. subtilis KDS was transformed with pBAio-lumiBI and cultivated on LB agar plates containing 20 μg/ml kanamycin and 5 μg/ml chlorampenicol at 37° C. A single clone was picked and cultivated in 2 ml LB-Lennox media with 20 μg/ml kanamycin and 5 μg/ml chlorampenicol for 16 h at 37° C. with agitation. In parallel, B. licheniformis P308 was cultivated in 2 ml LB-Lennox media for 16 h at 37° C. with agitation. The next day, pBAio lumiBI was conjugated into B. licheniformis P308 as described Maya M, 2006., Biosci Biotechnol Biochem; 70(3):740-742.), the mixed cells were spread on minimal salt agar plates supplemented with 2% glucose, 0.2% potassium glutamate and, 20 μg/ml kanamycin and incubated at 37° C. for 3 days. The B. subtilis donor strain KDS with tryptophane and threonine auxotrophies can no longer grow on these agar plates. A single B. licheniformis clone carrying pBAio-lumiBI was picked and cultivated in 2 ml LB-Lennox media with 20 pg/ml kanamycin for 16 h at 37° C. with agitation. The next day, the culture was diluted to an OD600 of 0.1 in LB-Lennox media supplemented with 20 μg/ml kanamycin. 200 μl culture each was transferred to a black 96 well microtiter plate (Sarstedt) and supplemented with xylose to final concentrations of 0%, 0.0313%, 0.0625%, 0.125%, 0.25% or 0.5% xylose. The cells were incubated in a Synergy Neo2 HTS Multi-Mode Microplate Reader (BioTek) at 37° C. with medium agitation. The OD600 and luminescence were measured after 8 h of cultivation. The relative luminescence units (RLU) as luminescence signal per OD600 at the 8 h timepoint is determined and plotted against the indicated xylose concentrations (FIG. 7B). The change of plasmid copy number is reflected from the change of the luminescence output of Pveg-lux, encoded on plasmid pAio-lumiBI. Induction of cop expression by addition of 0.031% xylose inducer molecule results in 2.5-fold reduction of the RLU signal (40%) of the non-induced control. Stronger cop expression by increased concentration of xylose results in even lower constant RLU signal levels which correspond to single copy integrated plasmid pBAio-lumiBI into the amyB locus.

In parallel to the luminescence experiment, samples from the 0.0%. 0.0313% and 0.5% xylose cultivations at the 8 h timepoint were withdrawn and were recovered by centrifugation. Total DNA was isolated from the individual samples and the average plasmid copy number per cell determined as described under example 1, however with oligonucleotides SEQ ID NO. 81 and SEQ ID NO. 82 within the luxB gene of plasmid pBAio-lumiBli and oligonucleotides SEQ ID NO. 83 and SEQ ID NO. 84 within the gene BLi02076 (locus tag DSM13) in the vicinity of the chromosomal terminus were used for RT-qPCR amplification. The result is depicted in FIG. 7C. The average plasmid copy number of pBAio lumiBI in B. licheniformis without xylose inducer molecule was calculated to be 19.3 copies per cell. The lower average copy number compared to plasmid pKS100 (identical pE194 origin of replication with average 38.5 copies per cell; Example 1) reflects the leaky cop expression from promoter PxylA and hence repression of plasmid replication. The addition of 0.313% xylose results in an even lower average plasmid copy number of 11.6 per cell which is in agreement with the above described luminescence data (FIG. 7B). Strong cop expression by addition of 0.5% xylose leads to a calculated average plasmid copy number of 0.8 — slightly below 1.0 which indicates plasmid loss. Cells without plasmid are not viable under antibiotic selective pressure, however, contribute their genomic DNA as detected by qPCR analysis.

### Example 7

### 100% Campbell Recombination Efficiency by Combination of Overexpression of Cop in B. licheniformis and Shift to Non-Permissive Temperature

Homologous recombination-based gene deletion or gene integration approaches using plasmids with temperature sensitive origin of replications e.g. pE194, force first Campbell recombination of the plasmid into the genome with a homology region present on the plasmid by shifting cultivation conditions to the non-permissive temperature under selective pressure (e.g. antibiotics). This procedure is not complete and results in large amounts of cells that presumably have non-replicating plasmid at very low copy number, eventually leading to large screening efforts to identify the correct clones.

In order to show the higher efficiency of pBAio based plasmids with respect to the efficiency of homologous recombination with the host genome, a “light switch” was constructed. The luciferase encoding genes (luxABCDE reporter operon) under the control of the strong PsrfAA promoter from B. subtilis were integrated into the amylase amyB locus of B. licheniformis P308 with plasmid pKS068 creating B. licheniformis strain LBL as described under the strains section. Each cell of B. licheniformis LBL shows a luminescence signal. Plasmid pKS137, a pBAio plasmid derivative carrying a region homologous to 500 bp of luxA, was transformed by conjugation in B. licheniformis LBL as described in Example 6 (see above). One colony was picked and inoculated in 10 ml LB Lennox supplemented with 20 μg/ml kanamycin and 0.5% xylose and incubated at 37° C. with agitation for 3 h (pre-culture). A dilution series was made and 100 μl each are plated on LB agar plates containing 20 μg/ml kanamycin and 1% xylose. The agar plates were incubated over night at 45° C. As controls, cells were treated without xylose in the pre-culture and/or the agar plates, incubated at 37° C. or combinations thereof. The next day, colonies were examined for the presence or absence of luminescence to analyze the plasmid integration status. Agar plates were analyzed for luminescence by 3 min of exposition at default settings with the Alphalmager™ (Alpha Innotech). A second picture was taken with reflective light for 8 ms at the high-resolution setting. The two pictures were overlaid using Fiji (Schindelin et al., 2012, Nature methods 9(7): 676-682). The plasmid integration rate was determined by dividing the number of non-luminescent colonies by the total number of colonies. On average 1000 colonies per condition were analyzed and the experiment was performed in triplicates.

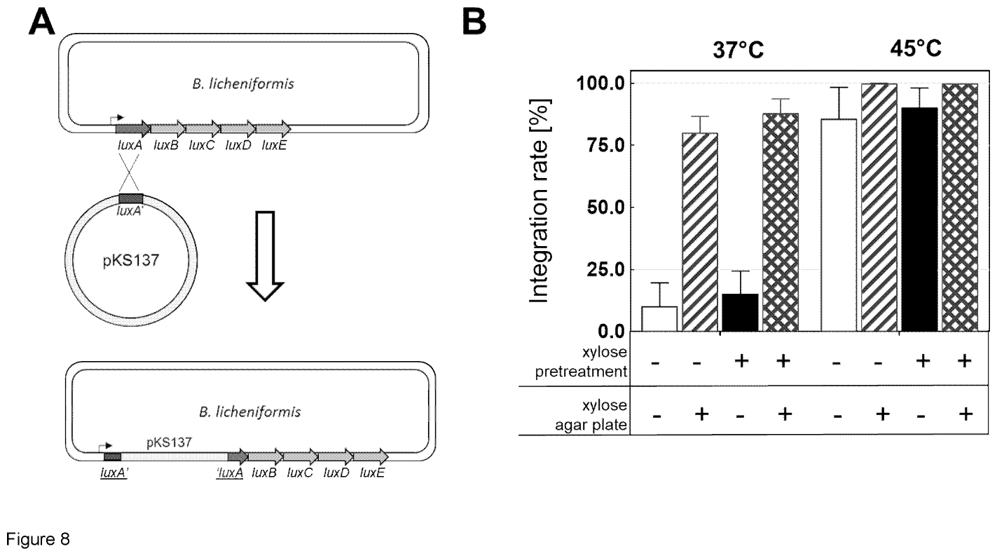

Clones with integrated plasmid do not show luminescence as the homologous recombination of plasmid pKS137 with the luxA gene results in integration of the plasmid into the genome and therefore disruption of the luxABCDE operon. A schematic representation is depicted in FIG. 8A and the results are depicted in FIG. 8B.

At 37° C. plasmid integration rates of up to 87% could be achieved by plating on LB agar plates containing 1% xylose and hence induction of cop expression. Induction of cop gene expression in liquid culture with ‘pretreatment’ by addition of 0.5% xylose exerts a minor effect on the overall integration efficiency. At 45° C. — the non-permissive temperature — the average integration rates are between 85% and 90% which demonstrates that the first step is indeed inefficient. Combination of growth at the non-permissive temperature for the pE194 origin of replication (45° C.) with induction of cop expression shows that the integration rate can be increased to 100% (no luminescent colony in over 1000 colonies analyzed).

### Example 8

### Suicide Vector and Method

The gene deletion (analogous gene integration) procedure applying temperature-sensitive replication origins—as also described herein for the pBAio plasmid with additional cop gene under the control of the inducible promoter PxylA—are used for genome editing, where the direct transfer of linear DNA fragments or suicide plasmids (not able to replicate in the host cell) comprising a selectable marker flanked on each side by DNA sequences homologous to the recipient cell is not successful as determined by transformants that have taken up the DNA and correctly integrated into the genome. The former has been numerously described for Bacillus licheniformis, Bacillus pumilus and other species, the latter has been described for B. subtilis laboratory strains (e.g. B. subtilis 168 strain) with so called natural competence where linear DNA is actively taken up by the cell concomitant with a high frequency of recombination. Likewise, ‘inducible competence’ systems have been described by plasmid-based or genomically integrated based overexpression of competence genes comS and/or comK to enhance DNA uptake. Also known is the additional knockout of the mecA gene in B. licheniformis rendering the host cell into a recipient host with improved transformability with linear DNA. Obvious, the recipient host needs to be genetically manipulated prior to be used. Moreover, detrimental effects on the host's production capability (e.g. chemicals, polymers, proteins, enzymes etc.) may occur. Therefore, it is desired to apply an efficient DNA transfer and gene deletion/gene integration procedure without the need of modification of the recipient cell.

The DNA transfer and gene deletion procedure comprises:
B. subtilis donor host with an auxtrophy (e.g. alanine racemase gene—deletion of the alr/dal gene) and the conjugative plasmid pLS20 (WO0200907). The conjugative plasmid pLS20 carries in addition a selectable marker (e.g. chloramphenicol resistance marker, ltaya et al. 2006) and a counterselection marker (e.g. the codBA genes Kostner et al 2013). The B. subtilis host is a derivative of the B. subtilis host Bs#056, where the chloramphenicol resistance gene had been replaced by a kanamycin resistance cassette flanked by lox sites which had subsequently been removed by transfer of cre recombinase on plasmid pDR244 (Bacillus Genetic Stock center BGSC: ECE274). The replication cassette of plasmid pE194 (nt 920-nt1910; comprising the cop and repF gene under control of the Pcop promoter as well as the antisense ctRNA) is integrated into the threonine (thrC) locus with a plasmid derivative pBS4S (Radeck et al. 2013) where the spectinomycin resistance cassette had been replaced by a kanamycin resistance cassette flanked by lox sites to allow for cre-mediated marker recycling. Preferentially an additional copy of the cop gene is placed under the inducible PxylA gene promoter integrated into the sacA locus to allow for adjustment of low-copy number of pE194 derivatives as described for B. subtilis GCX1.

The deletion plasmid is composed of the following elements:
A) Antibiotic resistance gene (e.g. kanamycin resistance gene of plasmid pUB110) and ColE1 replication; B) the oriT from pLS20 Maya M. et al., 2006, Bioscience, Biotechnology, and Biochemistry, 70(3), 740-742); C) the pE194 sequences comprising SSO and DSO (nt 630-nt 920); D) a counterselection marker (e.g. the codBA genes Kostner et al 2013); E) the spectinomycin resistance cassette flanked by lox sites and 5′ and 3′ 500 bp regions homologous to the regions of the recipient host.

The gene integration plasmid is composed of the following elements:
A) Antibiotic resistance gene (e.g. kanamycin resistance gene of plasmid pUB110) and ColE1 replication; B) the oriT from pLS20 Maya M. et al., 2006, Bioscience, Biotechnology, and Biochemistry, 70(3), 740-742); C) the pE194 sequences comprising SSO and DSO (nt 630-nt 920); D) a counterselection marker (e.g. the codBA genes Kostner et al 2013); E) the heterologous gene expression cassette (promoter-gene-terminator) which is located 5′ to the spectinomycin resistance cassette which is flanked by lox sites. The gene expression and spectinomycin resistance cassette is again flanked 5′ and 3′ 500 bp regions homologous to the regions of the recipient host.

Plasmid replication within the cell is dependent on repF protein encoded in trans on the chromosome or plasmid-based.

The Bacillus licheniformis recipient host being prototrophic for D-alanine racemase activity is insensitive against 5′fluoro-cytosine and sensitive against kanamycin and spectinomycin antibiotics.

The gene deletion plasmid is assembled via type-II-assembly and transformed into the B. subtilis donor strain following selection on LB-agar plates supplemented with 20 μg/ml kanamycin, 5 μg/ml chloramphenicol, 10 μg/ml threonine and 100 μg/ml D-alanine. B. subtilis clones carrying the correct assembled gene deletion plasmid are identified using colony PCR following plasmid isolation and restriction enzyme digest analysis and sequencing. The plasmid is stable as replicative plasmid as repF is expressed from the chromosome as described above.

Next, the deletion plasmid is transferred from B. subtilis donor strain to the B. licheniformis recipient strain via conjugation as essentially described in example of WO0200907 for replicative pE194 based gene deletion/gene integration plasmids. The conjugation is modified according to ltaya et al 2006 where conjugation is performed in liquid culture and not on solid media agar plates.

B. subtilis donor strain carrying the deletion plasmid is grown 15-17 h in LB-Lennox medium supplemented with 20 μg/ml kanamycin, 5 μg/ml chloramphenicol, 10 μg/ml threonine and 100 μg/ml D-alanine and B. licheniformis recipient strain is grown in LB-Lennox medium at 37° C. The next days the cultures are diluted in fresh prewarmed media to an optical density OD (600 nm) of 0.1 and cultivation is continued in medium without antibiotics until both donor and recipient cell cultivations reach an OD(600) of 0.8 to 1.0. 0.5 ml of each culture is withdrawn, mixed and incubated for 15 min at 37° C. without shaking to allow conjugative plasmid transfer. Cell mixtures are washed once with prewarmed transformation medium (TM) following plating on TM-agar plates supplemented with 200 μg/ml spectinomycin and incubation for 24-48 hours at 37° C.

Transformation medium TM consists of Spizizen minimal medium (Anagnostopoulos, C. and Spizizen, J. (1961). J. Bacteriol. 81, 741-746.) with 1.0% glucose 0.2% potassium glutamate and 0.1% casamino acids.

Only B. licheniformis transconjugant cells that have received the linear single stranded deletion plasmid and successfully integrated the spectinomycin resistance cassette with double homologous recombination with the two homologous DNA regions are able to grow under these conditions. Plasmid replication of the donor plasmid is no longer possible as the repF gene is not present in B. licheniformis, resulting in a conjugated ‘suicide’ plasmid.

B. subtilis donor cells cannot grow in the absence of D-alanine. B. licheniformis transconjugants are next plated on TM-agar plates with 200 μg/ml spectinomycin and 20 μg/ml fluoro-cytosine with incubation for 24 hours at 37° C. The second step counterselects for the presence of pLS20CAT conjugative plasmid, which is self-transmissible, and for deletion plasmid if integration via Campbell recombination occurred.

The resulting B. licheniformis strain is deleted for the target gene and is spectinomycin resistant, insensitive to 5-fluoro-cytosine, sensitive to kanamycin.

In a next step, the spectinomycin resistance marker can be removed by resolvase (creresolvase) mediated marker recycling.

In contrast to other physical methods of DNA transfer like electroporation or protoplast transformation, conjugative DNA transfer renders the recipient cell ready for DNA uptake and DNA recombination by this enabling specific gene deletion/gene integration via this suicide approach.

Moreover, this method allows to construct gene-expression cassettes under the control of strong promoters, where a very low copy number in the B. subtilis donor strain is absolutely necessary for stable maintenance. As outlined the plasmid copy number is controlled in trans via repF expression levels in the cell. Upon conjugative transfer, a suicide plasmid is created in the recipient cell and the strong gene expression cassette is directly integrated into the genome without a multi-copy plasmid intermediate which would lead to selection of cells with mutations within the gene expression cassette.

1.-16. (canceled)

17. A plasmid copy number control host system, comprising a prokaryotic host, wherein the prokaryotic host comprises a first plasmid, wherein the first plasmid includes
an origin of replication activatable by a plasmid replication initiator protein (Rep);
an expression cassette comprising a promoter, a first cop gene coding for a repressor of the promoter and a rep gene coding for the plasmid Rep; and
optionally on a helper plasmid, a further cop gene,
wherein the first or the further cop gene is under control of an inducible promoter.

18. The plasmid copy number control host system according to claim 17, wherein the first plasmid further comprises an integration sequence comprising a sequence for homologous recombination.

19. The plasmid copy number control host system according to claim 17, wherein the first plasmid further comprises a selectable marker.

20. The plasmid copy number control host system according to claim 17, wherein the rep gene codes for a thermolabile Rep such that replication of the first plasmid is reduced when the prokaryotic host is cultivated at or above a non-permissible temperature.

21. A method of plasmid copy number control comprising the steps of
i) providing the plasmid copy number control host system according to claim 17; and
ii) inducing expression of the further cop gene to reduce a plasmid copy number to a desired level.

22. An integration method comprising the steps of
i) providing the plasmid copy number control host system according to claim 17; and
ii) cultivating the plasmid copy number control host system at or above a non-permissible temperature in the presence of an inductor of the further cop gene and under selection pressure for a selectable marker.

23. The plasmid copy number control host system according to claim 17, wherein the prokaryotic host is of genus Bacillus.

24. The plasmid copy number control host system according to claim 17, wherein the further cop gene is under control of an inducible promoter.
